(19) United States
(12) Patent Application Publication
(10) Pub. No.: US 2014/0171356 A1
(43) Pub. Date: Jun. 19, 2014

(54) CHEMICALLY IMMOBILIZED WNT PROTEIN AND METHODS OF USE

(71) Applicant: The Board of Trustees of the Leland Stanford Junior University, Palo Alto, CA (US)

(72) Inventors: Shukry J. Habib, Stanford, CA (US); Roeland Nusse, Stanford, CA (US)

(21) Appl. No.: 14/105,040

(22) Filed: Dec. 12, 2013

Related U.S. Application Data
(60) Provisional application No. 61/736,441, filed on Dec. 12, 2012.

(51) Int. Cl. A61K 38/16

(52) U.S. Cl. CPC A61K 38/16
USPC 514/1.1; 435/375; 435/7.21; 530/402

(57) ABSTRACT

Methods are provided for contacting a target cell with a biologically active Wnt polypeptide that is coupled to a magnetic particle, for producing biologically active bead-coupled Wnt stimulator and Wnt inhibitor polypeptides, and for enriching a target cell population for Wnt responsive or DKK responsive cells. Compositions and kits for practicing the methods of the invention are also included, and generally include a biologically active Wnt stimulator polypeptide that is coupled to a magnetic particle. Compositions and kits may also include (i) a buffer that is substantially free of detergent, and/or (ii) a biologically active bead-coupled DKK polypeptide.

### BACKGROUND

Asymmetric cell division is a fundamental process involved in many aspects of cell biology, developmental biology and cancer. How cells internally organize to produce asymmetry and how asymmetric divisions orient in such a way that different daughter cells become correctly located in tissues are questions of vital importance. Cell-to-cell signaling is critically important during the arrangement of newly divided cells in tissues, but our understanding of external cues that control asymmetric divisions is limited.

Asymmetric division is also a crucial aspect of stem cell biology. During mitosis, stem cells can become polarized such that they partition cell fate determinants and orient the mitotic spindle to maintain stem cell numbers via self-renewal while generating differentiated daughter cells. Stem cell self-renewal is dependent on external signals, often those of the Wnt family. Secreted Wnt glycoproteins form a family of signaling molecules that trigger the highly conserved Wnt signaling pathway, which regulates numerous cell-to-cell interactions during embryogenesis and throughout adult life. The Wnt signaling pathway controls several aspects of cellular behavior, including fate choice, cell differentiation, cell growth, cell proliferation, cell survival, morphogenesis, organogenesis, and tissue patterning. In many tissues, abnormally decreased Wnt signaling leads to loss of stem cells while abnormally increased Wnt signaling can generate excess stem cell numbers, resulting in cancer.

Wnt proteins are hydrophobic owing to their modification by lipids, limiting the range of action of Wnt signals. This limited range is a hallmark of signals emanating from a stem cell niche, which is a local tissue environment controlling stem cell behavior. The limited range is also related to the polarization of target cells. Developmental signals, such as Wnts, are often presented to cells in a local manner from a particular direction. Wnts can induce different types of cellular responses, and these may depend on how cells read both the level and direction of the signal. The consequences of local signaling on dividing cells may range from orienting division to specifying asymmetric fates of daughter cells. However, the complexity of tissues and the multiplicity of signals that cells encounter create great challenges to understanding exactly how a particular localized growth factor can affect cell behaviors at the single cell level. In vitro studies provide excellent opportunities to follow single cells and their divisions but growth factors added to the tissue medium present signals in a non-oriented way.

Embryonic stem cells (ES cells or ESCs) provide a model system to understand how dividing stem cells make the choice between self-renewal and differentiation. ES cells divide rapidly in culture and cell fate choices can be monitored with reporter genes, conditions important for experimental approaches that rely on examining single cells and live imaging. Purified Wnt proteins that activate Wnt signaling maintain self-renewal of several types of stem cells including embryonic stem cells (ES cells, i.e., ESCs). Conversely, the inhibition of Wnt signaling leads to differentiation of ES cells towards epiblast stem cells (EpiSCs), which are characterized by decreased expression of pluripotency genes and increased expression of markers of the mouse epiblast.

While the in vitro and/or in vivo use of purified Wnt protein facilitates the precise control of signaling specificity, purified Wnt protein is not presented to a target cell from a defined and controlled direction, and thus does not mimic cell-to-cell signaling in vivo. The clinical and/or research use of purified Wnt proteins will benefit from a defined and controlled local source of Wnt proteins. In addition, because active Wnt proteins are hydrophobic, current Wnt purification protocols require the presence of detergent to keep the purified Wnt protein soluble and in a biologically active form. The required presence of detergent in purified formulations of Wnt limits the concentration of purified Wnt that can be applied to living cells because increased levels of detergent are toxic to most cell types. The present invention addresses these needs.

Publications

(1) Koneracka, M: Immobilization of Enzymes on Magnetic Particles; Book Series Methods in Biotechnology; ISSN 1940-6061 (Print) 1940-607X (Online); Volume 22; Pages 217-228.

(2) Willert et al., Nature. 2003 May 22;423(6938):448-52

### SUMMARY

Methods are provided for asymmetric contacting of a target cell with a biologically active Wnt stimulator or inhibitor polypeptide, wherein the polypeptide is coupled to a magnetic particle. In some embodiments, a target cell is asymmetrically contacted with a Wnt polypeptide. In some embodiments, a target cell is contacted with a Wnt stimulator polypeptide for a period of time sufficient to elicit Wnt pathway signaling activity. In some embodiments, the methods of asymmetric contacting result in asymmetric cell division where the two daughter cells exhibit different cell fates or different states of differentiation. In some embodiments, the methods of asymmetric contacting result in asymmetric cell division where one daughter cell exhibits decreased differentiation potential (i.e., the cell differentiates or begins differentiation) and the other daughter cell exhibits self-renewal (i.e., maintains its differentiation potential). Target cells of interest include any cells that may be responsive to a Wnt signal, e.g. pluripotent stem cells, cells positioned near sites of injury in vivo, and the like.

In some embodiments of the invention, the location of the magnetic particle is controlled by the location of a magnet. In some embodiments of the invention, the methods further comprise detecting the presence of Wnt pathway signaling activity by various methods known in the art, including for example determining the activity of a reporter construct, determining the expression status of a direct Wnt target gene, etc. In some embodiments of the invention, the methods further comprise evaluating a cellular effect of the elicited Wnt pathway signaling activity, including for example determining the absence or presence of a marker of pluripotency, determining the absence or presence of a marker of cellular differentiation, etc.

Methods are also provided for enriching a target cell population for Wnt responsive cells. In such methods, a target cell population is contacted with Wnt-coupled beads and a magnet is used to produce an isolated cell population.

Methods are also provided for producing biologically active bead-coupled Wnt stimulator and Wnt inhibitor polypeptides. In some embodiments, the method is a method of producing a biologically active bead-coupled Wnt polypeptide. In some embodiments, the method is a method of producing a biologically active bead-coupled DKK (Dickkopf) polypeptide. In some embodiments, activated beads are produced and mixed with a Wnt polypeptide or a DKK polypeptide. In some embodiments, bead-coupled Wnt polypeptides are at least 70% biologically active when stored in a buffer that is substantially free of detergent.

Compositions and kits for practicing the methods of the invention are also included, and generally include a biologically active Wnt stimulator polypeptide that is coupled to a magnetic particle. Compositions and kits may also include (i) a buffer that is substantially free of detergent, and/or (ii) a biologically active bead-coupled DKK polypeptide.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1 Wnt3a beads induce asymmetric distribution of components of the Wnt/β-catenin pathway. ES cells were co-cultured with Wnt3a beads (indicated by dashed yellow circle or reconstructed as a yellow oval) and immune-stained at various stages of cell division with LRP6 (white), APC (cyan) and β-catenin (red) antibodies. The beads are 2.8 μm.

FIG. 2 A-D Asymmetric inheritance of centrosomes and the orientation of the plane of mitotic division. (A) A schematic diagram of centrosome inheritance during cell division. (B) Single ES cells expressing EGFP-Ninein (cyan) and DsRedex-CETN1 (magenta) were co-cultured with Wn3a or Wnt5a beads and the division of the cell was monitored by time-lapse imaging. After the division, the cells were quantified based on the relative expression level of EGFP-Ninein. A fluorescent contaminant is indicated by a white asterisk. (C and D) ES cells expressing H2B-Venus were co-cultured with Wnt3a beads (blue) or Wnt5a beads (red) and the segregation of the chromosomes was followed by 3D time-lapse imaging. The orientation of the chromosome segregation (up to telophase) was quantified and presented in a pie chart. The yellow portion represents chromosome segregation away from the bead.

FIG. 3 A-E Transcriptional activity of the pluripotency gene Rex1 during ES cell division. Wnt3a (A) or Wnt5a (B) or DKK-1(C) or R-spondin-1 (D) beads were co-cultured with single Rex-1 GFP ES reporter cells. GFP localization was followed by time-lapse imaging. The endogenous protein localization was visualized with immunostaining of fixed cells. (E) The cell division under various conditions was quantified based on the relative expression of GFP. Red bar: higher GFP levels in the Wnt-proximal cell. Yellow bar: higher levels of GFP in the Wnt distal cell. Blue bar: similar levels of GFP in both cells. The location of the bead that contacted the cell during the division is marked by yellow circle.

FIG. 4 A-D Distal cells express markers of Epiblast stem cell fate. Wnt3a (A) or Wnt5a (B) beads were co-cultured with single cells of an Oct4-Venus ES cell line. Oct4-Venus was followed by time-lapse imaging. (C) Cell division was quantified based on the relative expression of Venus. (D) Single cells of LF2 female ES line were co cultured with Wnt3a or Wnt5a beads. After the division, the cells were fixed and immunostained with antibodies that recognize H3K27me3. Based on the relative expression of the H3K27me3, the dividing cells were sorted into categories for quantification. The location of the bead that contacted the cell during division is marked by yellow circle.

FIG. 5 Purified Wnt3a but not Wnt5a proteins support self-renewal of ES cells. Rex1-GFP ES cells were cultured for a week in serum free media in the presence of LIF and either purified Wnt3a or Wnt5a.

FIG. 6 A-D Biological activity of immobilized Wnt3a and Wnt5a proteins. (A) Purified Wnt3a or Wnt5a proteins were immobilized onto magnetic beads and visualized by immunostaining. (B) Wnt3a bead (black bars) but not vehicle bead (white bars) treatment for 24 hrs activates the SuperTopflash (STF) luciferase Wnt reporter in a dose-responsive manner. Error bars represent SD. (C) Wnt5a bead treatment inhibits soluble Wnt3a-induced STF reporter activation. (D) Wnt3a bead (black dots) treatment for 12 hrs activates the 7xTcf eGFP reporter in ES cells. The beads are 2.8 μm and can be used as a scale bar.

FIG. 7 A-C Inactivation of immobilized Wnt3a proteins by DTT treatment. (A) The chemistry of immobilizing purified Wnt proteins onto carboxylic acid beads. (B) Immobilized Wnt3a proteins were treated with different concentrations of DTT and the activity of the beads was assayed for SuperTopflash (STF) luciferase Wnt reporter activity of L cells. Error bars represent SD. (C) DTT treated Wnt3a beads (black dots) were incubated with 7xTcf eGFP reporter ES cells for 12 h and the number of eGFP positive cells was counted. The beads are 2.8 μm and can be used as a scale bar.

FIG. 8 Wnt5a beads do not induce asymmetric distribution of components of the Wnt/β-catenin pathway. ES cells were co-cultured with Wnt5a beads (indicated by dashed purple circle or reconstructed as a purple oval) and immuno-stained at various stages of cell division with APC (cyan), LRP6 (white) and β-catenin (red) antibodies. All fluorescent images are snapshots of 3D reconstruction of z stacks of the confocal images. Nuclear β-catenin is additionally visualized by representation in XYZ in the plane of the nucleus. The beads are 2.8 μm.

FIG. 9 The distribution of Frizzled-1(Fz1)-GFP during ES cell division. ES cells expressing Fz1-GFP were co-cultured with Wnt3a beads and the division was monitored by time-lapse imaging. The location of the bead (2.8 μm) that contacted the cell during the division is marked by dashed yellow circle.

FIG. 10 A-B The effect of Wnt beads on the distribution of marker proteins in ES cells. (A) Wnt beads were incubated with ES cells for 16 hrs. Then, cells were fixed and the distribution patterns of components of the Wnt/β-catenin in single cells (categories a-c) and post-mitotic cells (categories d-f) were examined. For Rex1-EGFP reporter ES cells, the effect was examined by live imaging. (B) The P-values for the categories (a) and (d) for the different proteins among different groups were calculated.

FIG. 11 A-C Expression of Nanog-Venus fusion protein in Wnt exposed cells. (A and B) Wnt3a beads or Wnt5a beads were incubated with single cells of the knock in Nanog -Venus ES cell line and the protein localization during cell division was monitored by time-lapse imaging . Selected frames are shown. The brightness of the signal for each frame was determined individually. The mean and SD of the Nanog-Venus signal intensity from each cell was quantified based on raw data. The signal intensity of the cell that retains contact with the bead after the division is represented with a red triangle. (C) Cell divisions were quantified based on the relative expression of Nanog-Venus in the prospective daughter cells. Red bar: higher Nanog-Venus levels in the Wnt-proximal cell. Blue bar: similar levels of Nanog-Venus in both cells. The location of the bead (2.8 μm) that contacted the cell during division is marked by dashed red circle.

FIG. 12 A-F Heterogeneous patterns of symmetric and asymmetric divisions in Nanog-Venus ES cells. (A to E) Wnt3a or Wnt5a beads were incubated with single Nanog-Venus ES cells and followed by time lapse imaging. The mean and SD of the Nanog-Venus signal intensity in each cell was quantified based on the relative expression of Nanog-Venus in the prospective daughter cells. (F) Quantification of two successive divisions. (A to F) The frame of the division of the cell that contact the bead is indicated by a red arrow. A turquoise arrow indicates the division frame of the Wnt-distal cell. The cell that contacts the bead after division is represented on the graph by blue “x”.

FIG. 13 A-B Transcriptional activity of pluripotency genes during ES cell division. (A and B) Wnt3a (a) or Wnt5a beads (b) were co-cultured with single ES cells harboring GFP reporters under the control of promoters of Sox2 (A) and Stella (B). GFP localization was followed by time-lapse imaging. Endogenous protein localization was visualized by immunostaining of fixed cells. The cell division was quantified (c) based on the relative expression of GFP. Red bar: higher GFP levels in the Wnt-proximal cell. Yellow bar: higher levels of GFP in the Wnt-distal cell. Blue bar: similar levels of GFP in both cells. The location of the bead that contacted the cell during the division is marked by dashed yellow circle.

FIG. 14 A-C ES cell division in the presence of multiple Wnt3a beads. Wnt3a beads were incubated with single cells of various pluripotency reporter lines and the division and the distribution of the beads to both daughter cells were followed by time lapse imaging. The location of the bead (2.8 μm) that contacted the cell during the division is marked by dashed yellow circle.

FIG. 15 A-B Biological activity of immobilized R-spondin-1 and DKK1. (A) Soluble and immobilized R-spondin-1 enhance the Wnt3a-mediated activation of SuperTopflash (STF) luciferase Wnt reporter in a dose-responsive manner. (B) Soluble and immobilized DKK-1 inhibit the Wnt3a-mediated activation of SuperTopflash (STF) luciferase Wnt reporter in a dose-responsive manner. Error bars represent SD.

FIG. 16 A-B Wnt-distal cells express markers of Epiblast stem cell fate. (A) and (B) Single cells of LF2 female ES line were co cultured with Wnt3a or Wnt5a beads. After the division, the cells were fixed and immunostained with antibodies that recognize Claudin6. Based on the relative expression of Claudin6, the dividing cells were sorted into categories for quantification. Red bar: higher Claudin6 levels in the Wnt-proximal cell. Yellow bar: higher levels of Claudin6 in the Wnt distal cell. Blue bar: similar levels of Claudin6 in both cell. The location of the bead that contacted the cell during the division is marked by dashed yellow circle. An arrow indicates the nanotube during cytokinesis (A).

FIG. 17 A scheme that can be used to enrich a population of target cells for Wnt responsive cells.

FIG. 18 Data collected while enriching a target cell population for Wnt responsive cells using the scheme depicted in FIG. 17. When the target cells were contacted with Vehicle only or with un-activated beads, none of the isolated cells were Wnt responsive (GFP+). When the target cells were contacted with Wnt3a-coupled beads, 70% of the isolated cells were Wnt responsive (GFP+).

### DETAILED DESCRIPTION OF THE EMBODIMENTS

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed

In the following description, reference will be made to various methodologies known to those of skill in the art of cell biology, stem cell biology, developmental biology, immunology, and molecular biology.

Methods

Embodiments of the invention include contacting a target cell with a biologically active Wnt polypeptide for a period of time sufficient to elicit Wnt pathway signaling activity, where the biologically active Wnt polypeptide is coupled to a magnetic particle.

Target Cells

The term “target cell” is a non-limiting term that is used herein to refer to any cell that can be contacted by a biologically active Wnt polypeptide of the subject methods (e.g., an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a human, etc.). A target cell can be any desired cell (e.g., a stem cell, e.g. an embryonic stem (ES) cell, an induced pluripotent stem cell (iPSC), a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. a stem cell, a progenitor cell, a terminally differentiated cell, etc.).

In some embodiments a target cell is in vitro (e.g., cultures of established cell lines, cultures of known or purchased cell lines, cultures of immortalized cells, cultures of primary cells, etc.). In vitro cells can be cells obtained from a subject (e.g., primary cell cultures, biopsies, tissue samples, whole blood, fractionated blood, hair, skin, and the like). In some embodiments a target cell is an isolated cell that is not in direct contact with a neighboring cell. By “not in direct contact with a neighboring cell” is meant that the target cell is not directly contacting (i.e., touching) a neighboring cell, although the cells may be present nearby (e.g., within 1 cell diameter, 2 cell diameters, 5 cell diameters, etc.). When a target cell is not in contact with a neighboring cell, the effect of contacting the target cell with a subject bead-coupled polypeptide can be determined without taking into account contact by another cell.

In some embodiments, a target cell is in vivo (e.g., a cell that resides in a living multi-cellular organism). An in vivo target cell can be located anywhere on or in the organism (i.e., the subject). In some embodiments, an in vivo target cell is located near a site of traumatic injury (e.g. a wound site).

In some embodiments, the Wnt stimulator or inhibitor polypeptide is removed after contacting the target cell (e.g., after Wnt signaling has been stimulated (i.e., elicited) or inhibited). For example, is a target cell is contacted, in vitro or vivo, by a subject magnetic bead-coupled Wnt stimulator or Wnt inhibitor polypeptide, a magnetic may be used to remove the beads.

Stem Cells

In some embodiments, the target cell is a stem cell. The term “stem cell” is used herein to refer to a cell that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into further restricted stem cells (e.g., Epiblast stem cells (described below), mesodermal stem cells, mesenchymal stem cells, and the like), which in turn can differentiate into cells that are further restricted (e.g., cardiomyocyte progenitors, neural progenitors, and the like), which can differentiate into end-stage cells (i.e., terminally differentiated cells, e.g., neurons, skeletal muscle cells, cardiomyocytes, adipocytes, osteoblasts, and the like), which play a characteristic role in a certain tissue type, and may or may not retain the capacity to proliferate further. Different types of stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to particular types of differentiated progeny.

The stem cells of interest can derive from any organism, e.g. a mammalian organism, where the term mammalian refers to a cell isolated from any animal classified as a mammal, including humans, domestic and farm animals, and zoo, laboratory, sports, or pet animals, such as dogs, horses, cats, cows, mice, rats, rabbits, etc. In some embodiments, the mammal is a human and the mammalian stem cell is therefore a human stem cell.

A “progenitor cell” is a type of stem cell that typically does not have extensive self-renewal capacity (i.e., the number of self-renewing divisions is limited), and often can only generate a limited number of differentiated cell types (e.g., a specific subset of cells found in the tissue from which they derive). Thus, a progenitor cell is differentiated relative to its mother stem cell, but can also give rise to cells that are further differentiated (e.g., terminally differentiated cells). For the purposes of the present invention, progenitor cells are those cells that are committed to a lineage of interest (e.g., a cardiomyocyte progenitor, a neural progenitor, etc.), but have not yet differentiated into a mature cell (e.g., a cardiomyocyte, a neuron, etc.).

When a stem cell divides symmetrically, both resulting daughter cells are equivalent. For example, a stem cell may undergo a self-renewing symmetric division in which both resulting daughter cells are stem cells with an equal amount of differentiation potential as the mother cell. However, a symmetric division is not necessarily a self-renewing division because both resulting daughter cells may instead be differentiated relative to the mother cell. When a stem cell divides asymmetrically, the resulting daughter cells are different than one another. For example, if a stem cell undergoes a self-renewing asymmetric division, then one of the resulting daughter cells is a stem cell with the same amount of differentiation potential as the mother cell while the other daughter cell is differentiated relative to the mother cell (e.g., a more lineage restricted progenitor cell, a terminally differentiated cell, etc.). A stem cell may directly differentiate (i.e., without dividing), or may instead produce a differentiated cell type through an asymmetric or symmetric cell division.

Stem cells (i.e., cell populations) of interest include “pluripotent stem cells” (PSCs, i.e., a PSC population). The term “pluripotent stem cell” or “PSC” is used herein to mean a stem cell capable of self-renewal and of producing all cell types of the organism (i.e., it is pluripotent). Therefore, a PSC can give rise to cells of all germ layers of the organism (e.g., the endoderm, mesoderm, and ectoderm). Pluripotent stem cells exist in two states: (i) a “naïve” state, which is epitomized by mouse embryonic stem cells (ESCs, described in more detail below) and (ii) a “primed” state, which is epitomized by the developmentally more advanced mouse epiblast stem cells (EpiSCs, described in more detail below). In the naive state, the PSC genome has an unusual open conformation and possesses a minimum of repressive epigenetic marks. In contrast, cells in the primed state have activated the epigenetic machinery that supports differentiation towards the cell types of the embryo. The transition from naive to primed pluripotency therefore represents a pivotal event in cellular differentiation. For more details regarding the naïve and primed states, see, for example, Nichols and Smith, Cell Stem Cell. 2009 Jun 5;4(6):487-92: Naive and primed pluripotent states.

PSCs may be in the form of an established cell line, they may be obtained directly from primary embryonic tissue, or they may be derived from a somatic cell. Because the term PSC refers to pluripotent stem cells regardless of their derivation, the term PSC encompasses the terms embryonic stem cell (ESC, described below), induced pluripotent stem cell (iPSC, described below), embryonic germ stem cell (EGSC, described below), and epiblast stem cells (EpiSC). A human PSC can be referred to as an “hPSC”, an “hESC”, an “hiPSC”, and the like, depending on the context and the derivation of the PSC. Likewise, a mouse PSC can be referred to as an “mPSC”, an “mESC”, an “miPSC”, an mEpiSC, and the like. The methods described herein are applicable to any mammalian PSC, including but not limited to an ESC, an iPSC, an EpiSC, and/or an EGSC.

During embryonic development, early cell proliferation in the pre-implantation embryo produces a sphere containing a cavity (i.e., blastocoel or blastocoel cavity) surrounded by a ring of trophoblast cells and an eccentrically located cell mass (the inner cell mass), which gives rise to the cells of the organism. The inner cell mass develops further into a structure composed of two layers: (i) a superficial cell layer called the epiblast and (ii) an inner cell layer called the hypoblast, which forms a border between the epiblast and the blastocoel cavity, and eventually expands to form the yolk sac. Once an embryo implants, it is considered a post-implantation embryo.

By “embryonic stem cell” or “ESC” it is meant a PSC derived from the inner cell mass of a pre-implantation embryo that is capable of dividing without differentiating (maintaining pluripotency) for a prolonged period in culture and is pluripotent (i.e., capable of giving rise to all cell types of the organism, e.g., cells of the three primary germ layers)(Thomson et. al, Science. 1998 November 6;282(5391):1145-7; Nichols and Smith, Cell Stem Cell. 2009 June 5;4(6):487-92). ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli.

In addition, ESCs exhibit markers of pluripotency known by one of ordinary skill in the art, including but not limited to Alkaline Phosphatase activity, SSEA3 expression, SSEA4 expression, Sox2 expression, Oct3/4 expression, Nanog expression, Stella/Dppa3 expression, TRA160 expression, TRA181 expression, TDGF 1 expression, Dnmt3b expression, FoxD3 expression, GDF3 expression, Cyp26a1 expression, TERT expression, Pecam1 expression, Tbx3 expression, Gbx2 expression, Daz1 expression, Stra8 expression, NrOb1/Dax1 expression, Fbxo15 expression, Piwil2 expression, Klf4 expression, Rex1/Zfp42 expression, and expression of certain miRNAs (e.g., see Jouneau et al., RNA. 2012 Feb;18(2):253-64. Epub 2011 Dec. 27). Examples of methods of generating and characterizing ESCs may be found in, for example, U.S. Pat. No. 7,029,913, U.S. Pat. No. 5,843,780, and U.S. Pat. No. 6,200,806, the disclosures of which are incorporated herein by reference. Methods for proliferating hESCs in the undifferentiated form are described in WO 99/20741, WO 01/51616, and WO 03/020920.

By “induced pluripotent stem cell” or “iPSC” it is meant a PSC that is derived from a cell that is not a PSC (i.e., from a cell this is differentiated relative to a PSC). iPSCs can be derived from multiple different cell types, including progenitor cells as well as terminally differentiated cells. (Takahashi et. al, Cell. 2007 Nov. 30;131(5):861-72; Takahashi et. al, Nat Protoc. 2007;2(12):3081-9; Yu et. al, Science. 2007 Dec. 21;318(5858):1917-20. Epub 2007 Nov. 20). iPSCs have an ES cell-like morphology, growing as flat colonies with large nucleo-cytoplasmic ratios, defined borders and prominent nuclei. In addition, like other PSCs, iPSCs exhibit one or more markers of pluripotency known by one of ordinary skill in the art, including but not limited to Alkaline Phosphatase activity, SSEA3 expression, SSEA4 expression, Sox2 expression, Oct3/4 expression, Nanog expression, etc. Examples of methods of generating and characterizing iPSCs may be found in, for example, U.S. Patent Publication Nos. US20090047263, US20090068742, U520090191159, US20090227032, US20090246875, and US20090304646, the disclosures of which are incorporated herein by reference.

Generally, to generate iPSCs, somatic cells are provided with a cocktail (i.e., combination) of reprogramming factors (selected from, for example, Oct3/4, SOX2, KLF4, MYC, Nanog, Lin28, etc.) known in the art to reprogram the somatic cells to become pluripotent stem cells. By “reprogramming factors” it is meant one or more, i.e. a cocktail, of biologically active factors that act on a cell to alter transcription, thereby reprogramming a cell to pluripotency. When reprogramming factors are provided to cells (i.e., cells are contacted with reprogramming factors), these reprogramming factors may be provided to the cells individually or as a single composition, that is, as a premixed composition, of reprogramming factors. The factors may be provided at the same molar ratio or at different molar ratios, and the factors may be provided once or multiple times in the course of culturing the cells.

By “embryonic germ stem cell” or “EGSC” or “embryonic germ cell” or “EG cell” it is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

Stem cells of interest further include “epiblast stem cells” or “EpiSCs.” Unlike an ESC, an EpiSC is derived from the epiblast layer, usually from a post-implantation embryo, and is generally considered to be in a primed state (i.e., can be considered to be farther along the developmental process than an ESC). Like ESCs, EpiSCs are considered to be pluripotent, as they form teratomas upon injection into immunocompromised mice. Unlike ESCs on the other hand, EpiSCs do not efficiently colonize host embryos (i.e., generate chimaeric mice) when injected into blastocysts. EpiSCs can, however, be converted to an ES-like state in which they efficiently participate in chimera formation and contribute to the germline. Examples of methods of generating and characterizing EpiSCs can be found in, for example, U.S. Pat. No. 8,153,423; U.S. Patent application number 20110088107; Tesar et. al, Nature. 2007 Jul. 12;448(7150):196-9. Epub 2007 Jun. 27; Brons et. al, Nature. 2007 Jul. 12;448(7150)1 91-5. Epub 2007 Jun. 27; and Najm et. al, Cell Stem Cell. 2011 Mar. 4;8(3):318-25, the disclosures of which are incorporated herein by reference.

Gene expression by mouse EpiSCs closely reflects their epiblast layer origin and is distinct from mouse ESCs. For example, an mEpiSC is characterized by (i) reduced expression of some ESC expressed genes (e.g., Pecam1, Tbx3, Gbx2, Rex1/Zfp42, Stella/Dppa3, Daz1, Stra8, NrOb1/Dax1, Fbxo15, Piwil2, Klf4 etc.), and certain miRNAs; (ii) maintenance of expression of some ESC expressed genes (e.g., Oct3/4, Cdh1/E-cadherin, Gdf3, Tdgf1, Myc, etc.), including certain miRNAs; and (iii) increased expression of some genes (e.g., Claudin6 (Cldn6), FGF5, Nodal, Otx2, Leftyl, Pitsx2, Acta2, Lefty2, Eomes, Dkk1, Foxa2, brachyury(T), Gata6, Sox17, Cer1, etc.), including certain miRNAs (Tesar et. al, Nature. 2007 Jul. 12;448(7150):196-9. Epub 2007 Jun. 27; Brons et. al, Nature. 2007 Jul. 12;448(7150)1 91-5. Epub 2007 Jun. 27; Jouneau et al., RNA. 2012 February;18(2):253-64. Epub 2011 Dec 27). As non-limiting examples of markers (other than gene expression alone) of an mEpiSC relative to an mESC: (i) focal accumulation of histone H3 trimethylated on lysine 27 (H3K27me3) marks an inactivated X chromosome, which is characteristic of mEpiSCs, but not of mESCs; (ii) mESCs exhibit Alkaline Phosphatase activity while mEpiSCs do not; and (iii) although Oct3/4 expression is maintained in mEpiSCs, the enhancer (genomic locus controlling expression) that is active shifts from the distal enhancer (active in mESCs) to the proximal enhancer (active in mEpiSCs) (Tesar et. al, Nature. 2007 Jul. 12;448(7150):196-9. Epub 2007 Jun. 27).

It is recognized herein that typically derived mouse and human ESCs (hESCs) are not generally considered in the art to be identical. Mouse ESCs (mESCs) typically require LIF and/or BMP4 for their derivation and self-renewal while human ESCs typically require Activin/Nodal and/or FGF signaling. It has been recently recognized in the art that mEpiSCs appear to require the same two factors (FGF and Activin/Nodal signaling) as hESCs. It is also generally believed in the art that typically derived mESCs represent PSCs in the naïve state while typically derived hESCs and mEpiSCs both represent PSCs in the primed state. As such, the gene expression profile and epigenetic profile (e.g., location of H3K4Me3 and H3K27Me3 marks at specific loci) of a typically derived hESC more closely resembles that of an mEpiSC than an mESC. However, depending on culture conditions and/or the presence or absence of particular transcription factors, cells can be interconverted between the naïve and primed states (i.e., biased toward the naïve or primed state).

Several groups reported the derivation of hESCs and hiPSCs with biological properties similar to those of mESCs (Buecker et al., Cell Stem Cell. 2010 Jun. 4;6(6):535-46; Hanna et al., Proc Natl Acad Sci U S A. 2010 May 18;107(20):9222-7. Epub 2010 May 4; Li et al., Cell Stem Cell. 2009 Jan. 9;4(1)1 6-9. Epub 2008 Dec. 18; Wang et al., Proc Natl Acad Sci U S A. 2011 Nov. 8;108(45):18283-8. Epub 2011 Oct. 11). These hESCs exhibited morphology, growth properties, expression profiles and signaling dependence that were comparable to those of mESCs, but they were not stable in the absence of genetic manipulations. The fact that culture conditions are sufficient to interconvert between pluripotent states, both in mESCs and in hESCs, indicates that plasticity in the pluripotent state is more widespread than was previously appreciated.

By “somatic cell” it is meant a diploid cell of an organism that is not a germ cell and is not a pluripotent embryonic stem cell. Thus, in the absence of experimental manipulation, a mammalian somatic cell does not ordinarily give rise to all types of cells in the body, although adult somatic stem cells do exist (e.g., lineage restricted progenitor cells). Mammalian adult somatic stem cells typically give rise only to cell types of the organs in which they reside. While some animals have been shown to contain adult somatic pluripotent stem cells (e.g., planarians, i.e., flatworms), such a cell has not yet been shown to exist in mammals, including humans (Wagner et. al, Science. 2011 May 13;332(6031):811-6).

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” it is meant a cell that has exited from mitosis, i.e., it is “quiescent”, i.e. it is no longer undergoing divisions. This post-mitotic state may be temporary (i.e. reversible) or it may be permanent. Many terminally differentiated cells are considered to be post-mitotic, although some terminally differentiated cells can become mitotic under particular circumstances (e.g., injury).

Wnt Signaling Pathway

A target cell that is “Wnt responsive” is a cell that can respond to the extracellular presence of a Wnt protein by triggering the Wnt signaling pathway. A Wnt responsive cell comprises components of the Wnt signaling pathway (described in more detail below), including a receptor (e.g., a Frizzled receptor) that can bind to Wnt proteins. Not all cells are Wnt responsive. In some embodiments, the target cell is Wnt responsive. In some embodiments the target cell is not Wnt responsive. In some embodiments, it is unknown whether the target cell is Wnt responsive. In some embodiments, it is known whether the target cell is Wnt responsive. In some embodiments, the target cell is part of a heterogeneous population of target cells (i.e., a heterogeneous target cell population) in which some cells are Wnt responsive and some cells are not Wnt responsive. In some embodiments, it is known which cells of a heterogeneous target cell population are Wnt responsive. In some embodiments, it is unknown which cells of a heterogeneous target cell population are Wnt responsive.

The misregulation of Wnt signaling components at various stages during embryogenesis leads to catastrophic developmental defects while misregulation in adults leads to various disease states, including cancer. There are two main branches of the Wnt signaling pathway: (1) the canonical β-Catenin dependent Wnt signaling pathway and (2) the non-canonical β-Catenin independent pathways, which include planar cell polarity (PCP) signaling as well as Calcium signaling (Gao, et. al, Cell Signal. 2010 May;22(5):717-27. Epub 2009 Dec. 13). As used herein, the terms “Wnt signaling” and “Wnt/β-Catenin signaling” are used interchangeably to refer to the canonical β-Catenin dependent Wnt signaling pathway. As such, a Wnt signaling stimulator (i.e., agonist) (e.g., Wnt3a) increases output from the β-Catenin dependent Wnt signaling pathway while a Wnt signaling inhibitor (i.e., antagonist) decreases output from the β-Catenin dependent Wnt signaling pathway.

Aspects of the invention include modulation of the Wnt signaling pathway by contacting a target cell with a bead-coupled (i.e., particle-coupled) Wnt stimulator or inhibitor polypeptide, thus either stimulating or inhibiting activity of the Wnt signaling pathway. Activation of the Wnt pathway culminates when the protein β-Catenin enters the cell nucleus (for recent review of the canonical β-Catenin dependent Wnt signaling pathway see Clevers et. al., Cell. 2012 Jun. 8;149(6):1192-205: Wnt/3-catenin signaling and disease). However, in the absence of Wnt signaling, free cytosolic β-Catenin is incorporated into a complex, known in the art as the β-Catenin destruction complex, which includes the proteins Axin, Adenomatous Polyposis Coli (APC), and glycogen synthase kinase (GSK-3β). Phosphorylation of β-Catenin by GSK-3β designates β-Catenin for the ubiquitin pathway and degradation (e.g., via βTRCP).

Transduction of the β-Catenin dependent Wnt signaling pathway (i.e., the Wnt signaling pathway) is triggered by the binding of secreted Wnt ligands to two distinct families of cell-surface receptors: the Frizzled (Fz) receptor family and the LDL-receptor-related protein (LRP) family (Akiyama, Cytokine Growth Factor Rev. 11:273-82 (2000)). This binding leads to the activation of Dishevelled (Dvl) proteins, which inhibit glycogen synthase kinase-3β (GSK-3β) activity (i.e., phosphorylation of β-Catenin), leading to the cytosolic stabilization of β-Catenin. Stabilized β-Catenin then enters the nucleus and associates with the TCF/LEF (T Cell-specific transcription Factor/Lymphoid Enhancer Factor) family of transcription factors to induce transcription of downstream target genes.

In the absence of Wnt signaling, cytosolic (and therefore nuclear) levels of β-Catenin are kept low by negative regulatory components of the pathway while in the presence of Wnt signaling, cytosolic (and therefore nuclear) levels of β-Catenin are stabilized by positive regulatory components of the pathway. For this reason, β-Catenin levels (e.g., monitored via Western blot) can provide insight into whether the Wnt signaling pathway of a cell has been stimulated or inhibited (e.g., increased levels of β-Catenin indicate increased signaling and decreased levels indicate decrease signaling). Likewise, β-Catenin levels in the nucleus (e.g., monitored via fluorescence microscopy, Western blot, etc.) can also be monitored to determine increased or decreased signaling.

By “positive regulatory components” of the Wnt pathway, it is meant proteins that function by enhancing (i.e., stimulating) the Wnt pathway, thus resulting in increased Wnt pathway signaling activity (i.e., increased Wnt pathway signaling output, e.g., increased target gene expression, increased reporter activity, increased levels of β-Catenin, etc.). Examples of known positive regulatory components of the Wnt pathway include, but are in no way limited to: Wnt (secreted, extracellular), Norrin (secreted, extracellular), R-spondin (secreted, extracellular), PORCN, Wls, Frizzled, LRP5 and LRP6, Tspan12, Lgr4, Lgr5, Lgr6, Dvl, β-Catenin, and TCF/LEF. A secreted positive regulatory component of the Wnt pathway (e.g., Wnt, Norrin, R-spondin, and the like) is referred to herein as a “Wnt stimulator polypeptide”.

By “negative regulatory components” of the Wnt pathway, it is meant proteins that function by antagonizing (i.e., inhibiting) the Wnt pathway, thus resulting in decreased pathway output (i.e., decreased Wnt pathway signaling output, e.g., decreased target gene expression, decreased reporter activity, decreased levels of β-Catenin, etc.). Examples of known negative regulatory components of the Wnt pathway include, but are in no way limited to: WIF, sFRP, DKK, Wnt5, Wnt11, Notum, WISE/SOST, Axin, APC, GSK-3β, CK1y, WTX, and βTrCP. A secreted negative regulatory component of the Wnt pathway is referred to herein as a “Wnt inhibitor polypeptide”.

Wnt inhibitor polypeptides (i.e., secreted negative regulatory components of the Wnt signaling pathway) include members of the WIF (Wnt inhibitory factor), sFRP (Secreted Frizzled Related Protein), DKK (Dickkopf), Notum, and WISE/SOST families, which interfere with the appropriate interactions among Wnt, Frizzled, and LRP proteins (Melkonyan et al., 1997, Proc Natl Acad Sci U S A 94(25):13636-41; Moon et al.,1997, Cell 88(6):725-8; Fedi et al., 1999, J Biol Chem 274(27):19465-72; Nusse, 2001, Nature 411(6835):255-6; Clevers et. al., Cell. 2012 Jun. 8;149(6):1192-205: Wnt/β-catenin signaling and disease). Although most Wnt polypeptides are Wnt stimulator polypeptides, certain Wnt polypeptides (e.g., Wnt5 and Wnt11) are Wnt inhibitor polypeptides (see working example 1). Wnt5 and Wnt11 have been demonstrated to stimulate non-canonical (non-β-catenin dependent) Wnt signaling and have also been demonstrated to inhibit canonical (β-catenin dependent) Wnt signaling. Thus, the term “Wnt polypeptide” encompasses some Wnt stimulator polypeptides as well as some Wnt inhibitor polypeptides.

Suitable Wnt polypeptides include, but are in no way limited to human Wnt polypeptides. Human Wnt proteins of interest in the present application include the following: Wnt-1 (GenBank Accession No. NM_{—}005430); Wnt-2 (GenBank Accession No. NM_{—}003391); Wnt-2B (Wnt-13) (GenBank Accession No. NM_{—}004185 (isoform 1), NM_{—}024494.2 (isoform 2)), Wnt-3 (RefSeq.: NM_{—}030753), Wnt3a (GenBank Accession No. NM_{—}033131), Wnt-4 (GenBank Accession No. NM_{—}030761), Wnt-5A (GenBank Accession No. NM_{—}003392), Wnt-5B (GenBank Accession No. NM_{—}032642), Wnt-6 (GenBank Accession No. NM_{—}006522), Wnt-7A (GenBank Accession No. NM_{—}004625), Wnt-7B (GenBank Accession No. NM_{—}058238), Wnt-8A (GenBank Accession No. NM_{—}058244), Wnt-8B (GenBank Accession No. NM_{—}003393), Wnt-9A (Wnt-14) (GenBank Accession No. NM_{—}003395), Wnt-9B (Wnt-15) (GenBank Accession No. NM_{—}003396), Wnt-10A (GenBank Accession No. NM_{—}025216), Wnt-10B (GenBank Accession No. NM_{—}003394), Wnt-11 (GenBank Accession No. NM_{—}004626), Wnt-16 (GenBank Accession No. NM_{—}016087)). Although each member has varying degrees of sequence identity with the family, all encode small (i.e., 39-46 kD), acylated, palmitoylated, secreted glycoproteins that contain 23-24 conserved cysteine residues whose spacing is highly conserved (McMahon, A P et al., Trends Genet. 1992; 8: 236-242; Miller, J R. Genome Biol. 2002; 3(1): 3001.1-3001.15). Other Wnt polypeptides of interest in the present invention include orthologs of the above from any mammal, including domestic and farm animals, and zoo, laboratory or pet animals, dogs, cats, cattle, horses, sheep, pigs, goats, rabbits, rats, mice, frogs, zebra fish, fruit fly, worm, etc.

Suitable DKK polypeptides (DKK polypeptides are an example of a Wnt inhibitor polypeptide) include, but are in no way limited to human DKK polypeptides. Human DKK proteins of interest in the present application include the following: DKK1 (UniProt: O94907), DKK2 (UniProt: Q9UBU2), DKK3 (UniProt: Q9UBP4), and DKK4 (UniProt: Q9UBT3). Although each member has varying degrees of sequence identity with the family, all comprise two Cysteine-rich domains (a DKK-type Cys-1 domain and a DKK-type Cys-2 domain), the more C-terminal of which (DKK-type Cys-2 domain) interacts with the LRP5 and/or LRP6 protein to inhibit Wnt signaling. Other suitable DKK polypeptides of interest in the present invention include orthologs of the above from any mammal, including domestic and farm animals, and zoo, laboratory or pet animals, dogs, cats, cattle, horses, sheep, pigs, goats, rabbits, rats, mice, frogs, zebra fish, fruit fly, worm, etc.

### Biological Activity and Detergent-free Buffer

The methods of the present invention provide for bead-coupled wnt stimulator and/or inhibitor polypeptide compositions that are biologically active. The term “biologically active” is used herein to refer to a bead-coupled polypeptide that retains the effector functions that are directly or indirectly caused or performed by native sequence polypeptides. As such, subject biologically active bead-coupled Wnt stimulator polypeptides (e.g., Wnt3a, Norrin, etc.) stimulate the Wnt signaling pathway while subject biologically active bead-coupled Wnt inhibitor polypeptides (e.g., Wnt5, Wnt11, DKK, sFRP, etc.) inhibit the Wnt signaling pathway. Effector functions of native sequence Wnt stimulator polypeptides include stabilization of β-catenin, stimulation of stem cell self-renewal, induction of target genes, and induction of Wnt pathway reporters. Effector functions of native sequence Wnt inhibitor polypeptides include de-stabilization of β-catenin, stimulation of stem cell differentiation, reduction of target gene expression, and reduction of Wnt pathway reporter expression.

Functional assays for the biological activity of a subject polypeptide-coupled bead can include measuring the steady state level of stabilized β-catenin (which can be measured by quantitatively or qualitatively (e.g., “increase” versus “decrease”) measuring the steady-state level of cytosolic and/or nuclear β-catenin via standard methodology such as Western blotting, ELISA, fluorescence microscopy etc.), measuring the level of stem cell self-renewal and/or differentiation when contacted with a stem cell population (which can be measured using markers of cell fate), measuring the expression (i.e., expression status) of target genes (e.g., in Xenopus animal cap assays, in cells in vivo, in cells in vitro, e.g., human teratocarcinoma cells, breast progenitor cells, 293 cells, L cells, HeLa cells, and the like), and/or measuring the expression (i.e., expression status) of a reporter construct in a cell (e.g. an exogenous expression vector that may or may not be integrated into the genome of the cell, comprising a Wnt responsive promoter that drives the expression of a detectable protein, e.g., a fluorescent protein such as GFP, or an enzyme such as β-galactosidase, alkaline phosphatase, luciferase, and the like) in an in vitro or in vivo assay. Methods of determining the expression status of a target gene or reporter are well known in the art and can be either qualitative (e.g., present versus absent, on versus off, etc.) or quantitative (e.g., relative number or abundance of transcripts or protein, absolute number of transcripts or protein, etc.). Expression can be measured (i.e., the expression status can be determined) by any convenient method (e.g., antibody stains, Western blot, in situ hybridization, RT-PCR, quantitative RT-PCR (qRT-PCR), etc.).

An exemplary Western blot assay for Wnt stimulator or inhibitor biological activity contacts bead-coupled polypeptides with an experimental population of cells (e.g., mouse L cells, breast progenitor cells, etc.) and uncoupled beads with a control population of cells. The experimental and control cells are cultured for a period of time sufficient to stabilize β-catenin, usually about 1 hour, and lysed. The cell lysates are resolved by SDS PAGE, then transferred to nitrocellulose and probed with antibodies specific for β-catenin.

An exemplary reporter assay for Wnt stimulator or inhibitor biological activity includes contacting bead-coupled polypeptides with an experimental population of cells (e.g., mouse L cells, breast progenitor cells, etc.) that contain an exogenous expression vector that may or may not be integrated into the genome of the cell. The expression vector comprises a Wnt responsive promoter (e.g., a promoter having multiple TCF/LEF binding sites) that drives the expression of a detectable protein (e.g., a fluorescent protein such as GFP, RFP, CFP, YFP and the like; or an enzyme such as β-galactosidase, alkaline phosphatase, luciferase and the like). The assay further includes contacting uncoupled control beads with a control population of cells (the same cell type containing the same expression vector as the experimental population). The experimental and control cells are cultured for a period of time sufficient to induce reporter expression, usually about 30 minutes to 24 hours. For both groups of cells, reporter activity (e.g., fluorescence, enzymatic activity, etc.) is measured prior to contact with the beads as well as after contact with the beads to determine whether the beads stimulated or inhibited Wnt signaling activity. Optionally, reporter activity can be measured at the single cell level (e.g., via fluorescence microscopy) to determine whether Wnt signaling is stimulated or inhibited in bead proximal versus bead distal cells before, during, and/or after cell division.

Due to the fact that biologically active Wnt polypeptides are lipid modified and are therefore hydrophobic, a purified Wnt polypeptide normally requires a storage buffer that includes detergent to maintain biological activity (e.g., keep the polypeptide from precipitating out of solution). However, the inventors have discovered that bead-coupled Wnt polypeptides do not require detergent to maintain biological activity.

The term “substantially free of detergent” as used herein means free of detergent (e.g., detergent-free buffer), with the exception of small amounts of residual and/or contaminating detergent that may be present (e.g., the presence of detergent may be unknown to the practitioner). For example, a buffer that is substantially free of detergent contains 0.01% or less (e.g., 0.001% or less, 0.0001% or less, 0.00001% or less, or 0%) detergent. As used herein, the term “detergent-free buffer” means a buffer that is substantially free of detergent. The detergent-free buffer can be any buffer (e.g., phosphate buffered saline (PBS) at pH 7.4) in which a subject bead-coupled polypeptide retains biological activity. In some embodiments, the buffer also contains bovine serum albumin (BSA)(e.g., 0.5%, 1% BSA, 2% BSA, etc.).

In some embodiments, a subject bead-coupled polypeptide is 70% or more (e.g., 80% or more, 85% or more, 90% or more, 95% or more, 98% or more, 99% or more, or 100%) biologically active when stored in a detergent-free buffer (i.e., a buffer that is substantially free of detergent). As alluded to above, the inventors have discovered that bead-coupled Wnt polypeptides maintain their biological activity when stored in detergent-free buffer. Because a Wnt polypeptide can be a Wnt stimulator polypeptide (e.g., a Wnt polypeptide other than Wnt5 or Wnt11) or a Wnt inhibitor polypeptide (e.g., Wnt5 or Wnt11), the biological activity can be Wnt pathway stimulating biological activity or Wnt pathway inhibiting biological activity depending on the identity of the bead-coupled Wnt polypeptide.

One may determine the percent of biological activity when stored in a detergent-free buffer by comparing the biological activity, as measured using any of the techniques described above (e.g., Western blot, reporter activity, target gene expression, etc.), of a bead-coupled Wnt polypeptide stored in a detergent-free buffer to the biological activity of the freshly prepared bead-coupled Wnt polypeptide. For example, if so desired, one can measure the biological activity of a freshly prepared batch of bead-coupled Wnt polypeptide, and again measure the biological activity at various times (e.g., 1 hour, 2 hours, 6 hours, 12 hours, 24 hours, 2 days, 3 days, 5 days, 1 week, 2 weeks, 3 weeks, 1 month, 2 months, 6 months, 12 months, 18 months, 24 months, etc.) after storing the bead-coupled polypeptide in a detergent-free buffer. One can therefore monitor whether (and if so, how much) a stored bead-coupled polypeptide exhibits diminished biological activity. The absolute values measured by some assays can vary from experiment to experiment, which has the potential to render the measurement of biological activity at various time points unreliable. To account for this, one can utilize a standard Wnt pathway stimulator and/or or inhibitor (e.g., a purified Wnt polypeptide of known activity stored in the presence of detergent, a compound known to stimulate or inhibit the Wnt pathway, etc.) for comparison and standardization of experimental results.

One may determine the specific activity of a bead-coupled polypeptide by determining the level of activity in a functional assay (e.g., stimulation of target gene expression, upregulation of stem cell proliferation, β-catenin stabilization etc.) after administration by quantitating the total amount of bead-coupled protein present in a non-functional assay (e.g., immunostaining, Western blot, ELISA, quantitation on coomasie or silver stained gel, etc.), and determining the ratio of biologically active bead-coupled protein to the total amount of bead-coupled protein.

The bead-coupled Wnt polypeptide compositions find use in a variety of methods, including the maintenance and growth of stem cells, tissue regeneration, and the like. The ability to maintain Wnt polypeptide activity in a detergent-free buffer when the Wnt polypeptide is bead-coupled allows for the use of increased concentrations of biologically active Wnt polypeptide as compared to the concentration that can be used when administering a purified Wnt polypeptide stored in a detergent-containing buffer. This is due to the fact that detergents exhibit cellular toxicity above certain concentrations, which would limit the concentration of protein that can be administered to a cell when the Wnt polypeptide must be stored in a detergent-containing buffer. Thus, biologically active Wnt polypeptides can be administered to cells in higher concentrations when the Wnt polypeptide is coupled to a bead.

Detecting the Presence of Wnt Pathway Signaling Activity

Aspects of the subject methods include detecting the presence of Wnt pathway signaling activity. To detect the presence of Wnt pathway signaling, any of the assays discussed above for detecting the biological activity of a subject polypeptide-coupled bead can be used. Examples include, but are in no way limited to: detecting the steady-state level of cytosolic and/or nuclear β-catenin via standard methodology such as Western blotting, ELISA, fluorescence microscopy etc.; detecting stem cell self-renewal and/or differentiation using markers of pluripotency and/or cell fate; detecting the expression of direct Wnt target genes (described below) using techniques such as in situ hybridization, PCR (Polymerase Chain Reaction), quantitative RT-PCR (reverse transcriptase PCR), microarrays, etc.; and/or detecting the expression of a reporter construct (comprising a Wnt responsive transcriptional element that drives the expression of a detectable protein) in an in vitro or in vivo cellular assay.

In some embodiments, detecting the presence of Wnt pathway signaling activity is qualitative such that a “stimulated” versus “unchanged” versus “inhibited” decision can be made. Qualitative detection is suitable when a difference in signal can be detected in the presence versus the absence of a subject bead-coupled polypeptide. For example, in some cases, the target cell (or its daughter cell after division) does not exhibit detectable Wnt signaling activity or exhibits barely detectable Wnt signaling activity (e.g., a fluorescent or enzymatic reporter is undetectable or barely detectable, a direct Wnt target gene is undetectable or barely detectable, etc.) in the absence of a subject bead-coupled Wnt stimulator polypeptide, but does exhibit a noticeable detectable signal in the presence of a subject bead-coupled Wnt stimulator polypeptide. In some cases, the target cell exhibits noticeable detectable Wnt signaling activity in the absence of a subject bead-coupled Wnt inhibitor polypeptide, but does not exhibit (or exhibit barely detectable) Wnt signaling activity in the presence of a subject bead-coupled Wnt inhibitor polypeptide. As long as the difference in the amount of detectable signal representing Wnt signaling activity, between the presence and absence of a subject bead-coupled polypeptide, is large enough to detect and interpret, the detecting can be qualitative.

In some embodiments, detecting the presence of Wnt pathway signaling activity is quantitative such that the signal representing Wnt pathway signaling activity (e.g., the level of detectable reporter, e.g. fluorescent protein, enzymatic activity, etc.; the level of target gene expression; the nuclear level of β-catenin; etc.) is quantitatively determined in the presence and in the absence of a subject bead-coupled polypeptide. Thus, it can be quantitatively determined whether (and how much) Wnt pathway signaling activity has increased, decreased, or stayed the same in the presence versus the absence of a subject bead-coupled polypeptide.

Optionally, Wnt pathway signaling activity can be detected at the single cell level (e.g., via microscopy, e.g., fluorescence microscopy to detect reporters, fluorescence microscopy to detect the immunostaining of proteins encoded by target genes or to detect the immunostaining of marker proteins, brightfield microscopy to detect target gene expression after in situ hybridization or in situ PCR, brightfield microscopy to detect the enzymatic activity of a reporter enzyme, etc.) to determine whether Wnt signaling is stimulated or inhibited in individual bead-contacted cells. In some such cases, such detection methods can be used to determine the presence or absence of (or level of) Wnt signaling activity in bead proximal versus bead distal cells before, during, and/or after cell division.

Wnt target genes in various cells and tissues are known in the art and a list of examples (along with the references that describe each target) can be found, for example, at “www” followed by “stanford.edu/group/nusselab/cgi-bin/wnt/target genes.” The expression of some genes is increased (i.e., “induced”) while the expression of other genes is decreased (i.e., “repressed”) by stimulation of the Wnt pathway. Direct Wnt target genes are those genes whose expression is controlled (induced or repressed) as a direct result of the level of Wnt signaling activity (e.g., this usually means genes whose expression is controlled by control elements (e.g., promoters, enhancers, etc.) that directly respond to TCF/LEF binding). Examples of direct Wnt target genes include, but are not limited to: c-myc, Tcf-1, LEF1, PPAR delta, c-jun, fra-1, matrix metalloproteinase MMP-7, Axin-2, Nr-CAM, ITF-2, claudin-1, VEGF, FGF18, c-myc binding protein, Id2, Telomerase, LGR5/GPR49, Frizzled 7, Follistatin, Siamois, fibronectin, engrailed-2, Xnr3, connexin43, twin, dharma/bozozok, MITF/nacre, Brachyury, Osteocalcin, neurogenin 1 , SPS, NeuroD1, Nkx2.2, Gbx2, Cdx1, Pitx2, E-cadherin, Keratin , movo1, P16ink4A, CTLA-4, FGF4, versican, Tnfrsf19, Dpp, and stripe.

As alluded to above, synthetic constructs can serve as a “reporter” of Wnt signaling activity and such constructs are referred to as reporter constructs or Wnt reporter constructs. In a Wnt reporter construct, a reporter gene is operably linked to a transcriptional control element (e.g., promoter, enhancer, and the like) containing binding sites (usually multiple binding sites) for TCF/LEF such that the level of expression of the reporter gene, and therefore the level of activity of the encoded protein, is a direct read-out of the level of the activity of the Wnt signaling pathway. One exemplary Wnt reporter construct includes a TCF/LEF-driven luciferase reporter and the construct is referred to in the art as SuperTopFlash (STF). Another exemplary Wnt reporter construct is a TCF/LEF-driven eGFP (enhanced Green Fluorescent Protein) reporter and the construct is referred to as “7TCF//eGFP” (the “7” refers to the number of TCF binding sites in the transcriptional control element).

In some embodiments, a reporter is any convenient fluorescent protein such as GFP (Green Fluorescent Protein) or any variant thereof. For example, a reporter can be eGFP (enhanced GFP), CFP (cyan fluorescent protein), YFP (yellow fluorescent protein), RFP (Red fluorescent protein), DsRed, Venus, etc. For a review on some of the fluorescent proteins that can be used as a reporter, see “A guide to choosing fluorescent proteins” (Shaner et al, Nat Methods. 2005 December;2(12):905-9), which is incorporate herein by reference. In some embodiments, a reporter is an enzyme such as β-galactosidase, alkaline phosphatase, luciferase, and the like. Any Wnt reporter construct can be used in the subject methods to detect the level of Wnt pathway signaling activity as long as the transcriptional control element is responsive to canonical Wnt signaling and expression of the reporter is detectable.

Evaluating a Cellular Effect of Wnt Pathway Signaling Activity

Aspects of the subject methods include evaluating a cellular effect of Wnt pathway signaling activity. By “cellular effect” is meant a detectable change in the state of the cell (i.e., cellular state) that is not simply a change in expression of a target gene or reporter, but is instead a consequence of such a change. While a change in cellular state is not simply a change in expression of target genes, the change in expression of even a single gene can be indicative that a change in cellular state has occurred. For example, a gene (and/or the protein that it encodes) that is expressed only in differentiated cells can be used as a “marker” of cell differentiation while a gene (and/or the protein that it encodes) that is expressed only in pluripotent cells can be used as a “marker” of pluripotency. Thus, a change in expression of a single gene (or protein) that is a marker (e.g., of differentiation, of pluripotency, of a particular cell type, etc.) can indicate a change in cellular state. Therefore, detecting the expression of a marker gene (RNA or protein via any method, e.g., RT-PCR, in situ hybridization, Western blot, antibody staining, expression of a GFP-fusion, etc.) would be suitable for evaluating a cellular effect of Wnt pathway signaling activity.

Evaluating a cellular effect of Wnt pathway signaling activity can be performed with markers that are direct targets of Wnt signaling, with markers that are indirect targets, or with markers that are not considered to be “targets”. Indirect targets are genes (or their encoded proteins) that are not under direct transcriptional control by the Wnt pathway (e.g., they do not contain LEF/TCF binding sites in their transcriptional control element(s)), but whose expression does change as a secondary consequence of a stimulation and/or inhibition of Wnt signaling. For example, a transcription factor that is a directly induced target of Wnt signaling may secondarily induce or inhibit the expression of an additional gene. The additional gene in such a case would be considered an indirect target of Wnt signaling. An indirect target can be a repressed target (i.e., a target whose expression is decreased by active Wnt signaling) or an induced target (i.e., a target whose expression is increased by active Wnt signaling).

Any marker (e.g., a marker of cellular differentiation, a marker of pluripotency, a marker of a specific cell fate, etc.) that can be used to indicate a change in cell state that is an indicator of whether Wnt signaling has been modulated (i.e., whether signaling has increased, decreased, and/or remains unchanged) is suitable for the evaluation of a cellular effect of Wnt pathway signaling activity, regardless of whether it is a direct or an indirect target. Further, whether a particular marker is a direct or indirect target of Wnt signaling does not need to be known in order for the marker to be suitable in the subject methods.

In some embodiments, a marker used to evaluate a cellular effect of Wnt pathway signaling activity is an indirect target of Wnt signaling and thus marks the changes in the target cell that are a consequence of stimulated or inhibited Wnt signaling. For example, a gene (or it's encoded protein) that is expressed only in differentiated cells can be used as a marker of cellular differentiation even if the gene is not directly regulated by the Wnt signaling pathway. In such a case, if the inhibition of Wnt signaling activity causes differentiation from a pluripotent state, for example, then absence of the differentiation marker (or a decrease in levels of the marker) can indicate that Wnt signaling has remained the same or has been stimulated, depending on the experimental context (e.g., the state of the target cell prior to contact with a subject bead-coupled polypeptide). On the other hand, presence of the marker (or an increase in levels of the marker) can indicate that Wnt signaling has remained the same or has been inhibited, depending on the experimental context.

In some cases, a marker (direct or indirect target of Wnt signaling) is induced by Wnt signaling. As such, a detected increase in expression of the marker can indicate that Wnt signaling has been stimulated while a detected decrease in expression can indicate that Wnt signaling has been inhibited. In some cases, a marker (direct or indirect target of Wnt signaling) is repressed by Wnt signaling (i.e., not expressed or expressed at low levels in the presence of Wnt signaling). As such, a detected decrease in expression of the marker can indicate that Wnt signaling has been stimulated while a detected increase in expression (i.e., de-repression) can indicate that Wnt signaling has been inhibited.

As stated above, evaluating a cellular effect of Wnt pathway signaling activity can be performed with markers that are not usually considered to be “targets”. For example, evaluating a cellular effect of Wnt pathway signaling activity can be performed by monitoring changes in cellular morphology after contact with a subject bead-coupled polypeptide (Wnt stimulator or Wnt inhibitor polypeptide). For example, in some cases, cell morphology can indicate the state of differentiation of the target cell. While pluripotent cells tend to be round with a high nuclear to cytoplasmic volumetric ratio, differentiated cells (e.g., neurons, retinal cells, epithelial cells, etc.) can exhibit distinctive morphologies. As such, a change in morphology caused by contact with a subject bead-coupled polypeptide can be used to evaluate a cellular effect on Wnt pathway signaling activity (e.g., Wnt signaling stimulation versus inhibition, depending on the context).

Changes in epigenetic state are also suitable for evaluating a cellular effect of Wnt pathway signaling activity. Suitable examples include, but are in no way limited to: using markers of X-chromosome inactivation or activation, using markers of histone or DNA methylation (e.g., antibodies that detect H3K27Me3, H3K4Me3, and the like), etc. As an illustrative example, human female cells that have differentiated or have begun the differentiation process, and are therefore not pluripotent, exhibit inactivation of one of the X chromosomes. To the contrary, pluripotent cells do not exhibit X chromosome inactivation. Thus, a marker of X chromosome inactivation can serve as a marker of cellular differentiation and can therefore be suitable for the evaluation of a cellular effect of Wnt pathway signaling activity in contexts where Wnt signaling affects cellular differentiation.

In some embodiments, evaluating a cellular effect of Wnt pathway signaling activity is qualitative such that a “present” or “not present” decision can be made (e.g., when referring to the presence or absence of a marker of cellular state). Qualitative detection is suitable when a difference in cellular state can be detected in the presence versus the absence of a subject bead-coupled polypeptide. For example, in some cases, a target cell (or its daughter cell after division) exhibits a change in cellular state when the target cell is contacted with a subject bead-coupled polypeptide (bead coupled to a Wnt stimulator or Wnt inhibitor polypeptide). When comparing the presence versus the absence of a subject bead-coupled polypeptide, as long as the difference in the amount of detectable signal that represents cellular state (e.g., marker gene expression, cellular morphology, etc.), is large enough to detect and interpret, the evaluating can be qualitative. The absence and/or presence of a marker can be relative. For example, in some embodiments, the determination is based on comparing the activity or expression level of a marker in and/or on the cells in question with those of cells of a known fate (e.g., known pluripotent cells or known differentiated cells).

In some embodiments, evaluating a cellular effect of Wnt pathway signaling activity is quantitative such that the signal representing cellular state (e.g., the level of a marker, the localization of a protein in the cell, etc.) is quantitatively determined in the presence and in the absence of a subject bead-coupled polypeptide. Thus, it can be quantitatively determined whether (and how much) Wnt pathway signaling activity has changed the state of the target cell in the presence versus the absence of a subject bead-coupled polypeptide.

Optionally, evaluating a cellular effect of Wnt pathway signaling activity can be performed at the single cell level (e.g., via microscopy, e.g., fluorescence or brightfield microscopy to detect marker genes or proteins after in situ hybridization or immunostaining) to determine whether a change in cell state has occurred in individual bead-contacted cells. In some such cases, such detection methods can be used to determine the presence or absence of (or level of) a marker of cell state in bead proximal versus bead distal cells before, during, and/or after cell division.

In some embodiments, the marker of cell fate is a marker of pluripotency. In some embodiments, the marker of pluripotency is selected from a group consisting of: Alkaline Phosphatase (enzymatic) activity, SSEA3 expression, SSEA4 expression, Sox2 expression, Oct3/4 expression, Nanog expression, Stella/Dppa3 expression, TRA160 expression, TRA181 expression, TDGF 1 expression, Dnmt3b expression, FoxD3 expression, GDF3 expression, Cyp26a1 expression, TERT expression, Pecam1 expression, Tbx3 expression, Gbx2 expression, Daz1 expression, Stra8 expression, NrOb1/Dax1 expression, Fbxo15 expression, Piwil2 expression, Klf4 expression, Rex1/Zfp42 expression, and combinations thereof.

Alkaline phosphatase activity can be measured or tested by a variety of methods. For example, the cell in question can be contacted with a substrate for alkaline phosphatase that can be converted into a detectable (colorometric, fluorescent, etc.) molecule or state. The absence or presence of other markers of pluripotency (e.g., Oct 4 expression, nanog expression, etc.) can be determined by assaying for expression at either the RNA level (e.g., RT-PCR, microarray, etc. to determine mRNA) or the protein level (e.g., via antibody stain and FACS analysis or imaging).

In some embodiments, the presence of one or more (e.g., two or more, three or more, etc.) markers of pluripotency is indicative that the cell in question is pluripotent. In some embodiments, the absence of one or more (e.g., two or more, three or more, etc.) markers of pluripotency is indicative that the cell in question is not pluripotent (e.g., the cell is differentiated or has begun to differentiate).

Particles/Beads

Aspects of the invention include polypeptides that are coupled to particles (e.g., “magnetic beads”, “magnetic microparticles”, etc.). As used herein, the terms “particle”, “bead”, “microparticle”, and “microbead” are used herein interchangeably. Microparticles can be of any shape, and in some instances are approximately spherical (“microspheres”). Microparticles serve as solid supports or substrates to which other materials, such Wnt stimulator polypeptides or Wnt inhibitor polypeptides, can be coupled. A bead-coupled Wnt polypeptide can simply be referred to as a “Wnt-bead”, a “Wnt-particle”, or a “Wnt-microsphere”, all of which are used herein interchangeably. Likewise, a bead-coupled DKK polypeptide can simply be referred to as a “DKK-bead”, a “DKK-particle”, or a “DKK-microsphere”. Thus, a Wnt-bead and/or a DKK-bead can be referred to as such, or may be referred to in a more limited sense (e.g., a glass Wnt-bead, a magnetic Wnt-bead, a glass DKK-bead, a magnetic DKK-bead, etc.). Furthermore, a bead-coupled polypeptide may also be referred to as a polypeptide-coupled bead, which terms are used herein interchangeably (e.g., a magnetic bead-coupled Wnt polypeptide or a Wnt-coupled magnetic bead, etc.) Unless otherwise specified, a subject “bead-coupled polypeptide” is used herein to mean a bead that is coupled to a Wnt stimulator or Wnt inhibitor polypeptide.

A range of bead sizes are suitable for the methods, compositions, and kits provided herein. Beads can range in size from 0.01 to 1,000 μm (e.g., 0.1 to 100 μm, 1 to 100 μm, 1 to 10 μm, etc.) in diameter. In some embodiments, the beads can range in size from 2.5 to 3 μm (e.g., 2.7 to 2.9 μm, 2.5 μm, 2.6 μm, 2.7 μm, 2.8 μm, 2.9 μm, or 3.0 μm) in diameter. In some cases, it may be advantageous to use larger beads, (e.g., in some instances of performing cell separation when the fragility of the cells are not a concern). In some embodiments, the beads can range in size from 4.3 to 5.5 μm (e.g., 4.4-4.6 μm, 4.3 μm, 4.4 μm, 4.5 μm, 4.6 μm, 4.7 μm, 4.9-5.1 μm, 4.9 μm, 5.0 μm, 5.1 μm, 5.2 μm, 5.3 μm, 5.4 μm, or 5.5μm) in diameter.

Subject beads can be made of any convenient material (or combinations thereof), including, but not limited to inorganics such as metals, silica (e.g., SiO₂), glass, alumina, titania, ceramic, etc.; organics such as polystyrene, polymethylmethacrylate (PMMA); melamine, polyactide, etc.; and magnetic materials such as silica, polystyrene, dextran, etc. Commercially available magnetic beads include but are not limited to ProMag, COMPEL, BioMag, BioMagPlus, and Dynabeads. Microparticles in a variety of sizes and polymer compositions that are suitable for use in the preparation of subject bead-coupled Wnt stimulator polypeptides and/or bead-coupled Wnt inhibitor polypeptides are commercially available from a number of sources. Chemical monomers for preparation of microspheres are available from numerous sources. Microparticles can also be stained, e.g., with a fluorescent dye.

Compositions of, and methods of producing, suitable beads can be found in both the patent and non-patent scientific literature (e.g., U.S. Pat. No. 8,283,037: Magnetic microspheres for use in fluorescence-based applications; U.S. Pat. No. 5,597,531: Resuspendable coated magnetic particles and stable magnetic particle suspensions; U.S. Pat. No. 5,635,574: Microsphere and method for production thereof; and U.S. Pat. No. 8,163,183: Magnetic particle parallel processing apparatus permitting repeated use of container and method of magnetic particle parallel processing permitting repeated use of container), which are incorporated herein by reference.

Producing a Biologically Active Wnt Stimulator or Wnt Inhibitor Bead-Coupled Polypeptide

Aspects of the invention include methods of producing a biologically active bead that is coupled to a Wnt stimulator polypeptide or Wnt inhibitor polypeptide. A bead-coupled Wnt stimulator or inhibitor polypeptide can be referred to as an immobilized Wnt stimulator or inhibitor polypeptide. In some embodiments, the method is a method of producing a bead-couple Wnt polypeptide. Proteins can be covalently coupled to beads via carboxylic acid or other functional groups (e.g. amine groups (NH₂), aldehyde groups, etc.) on the surface of the beads. In some embodiments, a Wnt polypeptide is coupled to a bead that is coated with a carboxylic acid. In some embodiments, the coupling is performed using any convenient coupling agent (or combinations thereof). Suitable examples of coupling agents include, but are in no way limited to a carbodiimide (e.g., 1-ethyl-3-(3-dimethylaminopropyl) carbodiimide hydrochloride; 1-[3-(dimethylamino) propyl]-3-ethylcarbodiimide hydrochloride; 1-cyclohexyl-3-(2-morpholinoethyl) carbodiimide metho-p-toluene sulfonate; N-cyclohexyl-N′-(2-morpholinoethyl) carbodiimide methyl-p-toluensulfonate; and the like); NHS (N-hydroxysuccinimide); and the like. In some embodiments, a carbodiimide and NHS are both used as coupling agents.

A bead that will couple to a subject Wnt stimulator polypeptide or Wnt inhibitor polypeptide is referred to herein as an “activated” bead. In some embodiments, a carboxylic acid coated bead is contacted with a coupling agent (e.g., a carbodiimide, NHS, and the like) or multiple coupling agents (e.g., a carbodiimide and NHS) to produce an activated bead. In some embodiments, a bead (e.g., an aldehyde coated bead, e.g., an aldehyde coated glass bead) can be considered an activated bead without contacting a coupling agent.

The Wnt stimulator polypeptides and Wnt inhibitor polypeptides to be coupled to a bead can be produced from any source. Suitable sources include, but are by no means limited to: heterologous expression in Drosophila S2 cells, heterologous expression in vertebrate tissue culture cells (e.g. HEK293 cells, CHO cells, COS cells, NIH-3T3 cells, and the like), heterologous expression in bacterial cells, etc. Suitable polypeptides include those recovered from the culture medium as a secreted polypeptide or those recovered from host cell lysates. Some suitable Wnt stimulator polypeptides and Wnt inhibitor polypeptides are also available for purchase from commercial sources.

In some embodiments, a Wnt polypeptide (e.g., Wnt3, Wnt5, Wnt11, etc.) is recovered from culture medium as a secreted form of a Wnt protein. Wnt proteins have been found to be unexpectedly hydrophobic, due to the lipid modification. As such, a Wnt polypeptide (to be coupled to a bead) can be purified in the presence of a detergent to maintain solubility. Suitable detergents for this purpose include non-anionic detergents, and zwitterionic detergents, which may be used at a concentration of from about 0.25% to about 2.5%, usually at a concentration of from about 0.5% to 1.5%, and preferably at a concentration of about 1%. Non-anionic detergents include the Triton™ family of detergents, e.g. Triton™ X-15; Triton™ X-35; Triton™ X-45; Triton™ X-100; Triton™ X-102; Triton™ X-114; Triton™ X-165, etc. All of these heterogeneous detergents have a branched 8-carbon chain attached to an aromatic ring. This portion of the molecule contributes most of the hydrophobic nature of the detergent. Triton™ X-100 and NP-40 are very similar in structure and hydrophobicity and are interchangeable in most applications including cell lysis. Brij™ detergents are also similar in structure to Triton™ X detergents in that they have varying lengths of polyoxyethylene chains attached to a hydrophobic chain. However, unlike Triton™ X detergents, the Brij™ detergents do not have an aromatic ring and the length of the carbon chains can vary. Brij™ 58 is most similar to Triton™ X 100 in its hydrophobic/hydrophilic characteristics. The Tween™ detergents are nondenaturing, nonionic detergents, which are polyoxyethylene sorbitan esters of fatty acids. Tween™ 80 is derived from oleic acid with a C18 chain while Tween™ 20 is derived from lauric acid with a C12 chain.

The zwitterionic detergent, CHAPS, is a sulfobetaine derivative of cholic acid. This zwitterionic detergent is useful for membrane protein solubilization when protein activity is important. This detergent is useful over a wide range of pH (pH 2-12) and is easily removed from solution by dialysis due to high CMCs (8-10 mM).

In one embodiment of the invention, the Wnt protein is produced in a palmitoylated form. As described above, the presence of the palmitate causes Wnt to be relatively insoluble, and so isolation steps are preferably performed in buffer containing a concentration of detergent sufficient to maintain solubility. A first step in purification is dye ligand chromatography. The purified protein fraction can then be further purified by size exclusion chromatography, and by cation exchange chromatography. Purification of a Wnt polypeptide using such methods provides for a substantially homogeneous composition of biologically active Wnt protein. In some embodiments a Wnt polypeptide to be coupled to a bead is purified by Blue Sepharose affinity and gel filtration chromatography as described (Willert et al., Nature. 2003 May 22;423(6938):448-52: incorporated herein by reference).

In some cases, a subject polypeptide is concentrated prior to coupling. Concentration can be achieved using a number of methodologies, including but not limited to centrifugation using a cellulose membrane concentrator, dialysis, protein precipitation (i.e., salting out), chromatography, etc. In some embodiments a centrifugation using a Centricon® filter is used to concentrate a subject polypeptide prior to bead-coupling. In some embodiments, the subject polypeptide is a Wnt polypeptide and the desired protein concentration prior to coupling ranges from 100-300 ng/μl (e.g., 110-250 ng/μl, 130-200 ng/μl).

Biological activity of a purified Wnt stimulator polypeptide or Wnt inhibitor polypeptide can be confirmed via assays as described above (e.g., luciferase reporter assays using L cells stably transfected with the SuperTOPFlash reporter as described in Mikels and Nusse, PLoS Biol. 2006 April;4(4):e115: incorporated herein by reference).

In some embodiments, a Wnt stimulator polypeptide or Wnt inhibitor polypeptide is coupled to a bead having carboxylic acid groups. In some embodiments, the carboxylic acid groups are activated by incubating beads with a carbodiimide (examples are provided above) and with NHS (N-hydroxyl succinimide). After activation, the activated beads can be washed remove excess carbodiimide and NHS.

To couple a Wnt stimulator polypeptide or Wnt inhibitor polypeptide to an activated beads, the purified polypeptide to be couple is contacted (e.g., at room temperature) with activated beads for an appropriate amount of time. Appropriate times for contacting a Wnt stimulator polypeptide or Wnt inhibitor polypeptide with an activated bead to achieve coupling can range from 30 seconds to 10 hours (e.g., greater than 1 minute, greater than 5 minutes, greater than 10 minutes, greater than 20 minutes, greater than 30 minutes, greater than 1 hour, greater than 2 hours, greater than 4 hours, greater than 8 hours, for 10 hours, less than 10 hours, less than 8 hours, less than 4 hours, less than 2 hours, less than 1 hour, less than 30 minutes, less than 20 minutes, less than 10 minutes, less than 5 minutes, less than 3 minutes, less than 2 minutes, less than 1 minute, for 30 seconds, 30 seconds to 2 minutes, 1-2 minutes, 5 minutes to 8 hours, 30 minutes to 6 hours, 45 minutes to 4 hours, 1-4 hours, 45 minutes, 1 hour, 2 hours, 3 hours, or 4 hours).

In some embodiments, the methods include the use of a low pH buffer (e.g., a buffer that buffers at or near pH 5). A low pH buffer can be used at any appropriate step, for example, during any bead activation step, during a coupling step (e.g., when contacting an activated bead with a subject Wnt stimulator or inhibitor polypeptide), during a washing step, etc. In some embodiments, the buffer is MES (2-(N-morpholino)ethanesulfonic acid).

Once a subject bead-coupled Wnt stimulator or inhibitor polypeptide is produced, the bead-coupled polypeptide can be stored in any suitable detergent-free buffer (e.g., phosphate buffered saline (PBS) at pH 7.4) in which the bead-coupled polypeptide maintains biological activity. In some embodiments, the buffer also contains bovine serum albumin (BSA)(e.g., 0.5%, 1% BSA, 2% BSA, etc.). The subject bead-coupled polypeptides are generally stored at 4° C. although they may be stored at any temperature at which they retain biological activity for the desired amount of storage time. The amount of retained biological activity for any given stored bead-coupled Wnt stimulator or inhibitor polypeptide can be determined by measuring (i.e., determining) the biological activity as described above.

Enriching a Target Cell Population for Wnt Responsive Cells

Currently, it is difficult to purify Wnt responsive cells unless the cells have been rendered detectable. For example, cells genetically modified to incorporate a Wnt responsive promoter driving a reporter (e.g., a luciferase enzyme, a fluorescent protein, and the like) can be isolated and/or enriched by targeting cells based on the activity of the reporter (e.g., using fluorescent imaging, fluorescence activated cell sorting, etc.). However, such techniques do not allow for the isolation and/or enrichment of naturally occurring Wnt responsive cells because these methodologies require that Wnt responsive cells have been modified in some way (e.g., genetically modified). The subject methods, compositions, and kits can be used to isolate and/or enrich for Wnt responsive cells because Wnt responsive cells bind to the subject Wnt-coupled beads with high affinity. In other words, Wnt responsive cells can be said to specifically bind to the subject Wnt-coupled beads while Wnt non-responsive cells do not specifically bind to the subject Wnt-coupled beads.

Provided for are methods of enriching a target cell population. In some embodiments, the methods are methods of enriching a target cell population for Wnt responsive cells. In some embodiments, the methods are methods of enriching a target cell population for DKK responsive cells. Methods of enriching a target cell population include contacting the target cell population with a subject bead-coupled polypeptide, isolating the beads to produce an isolated cell population comprising cells bound to the beads (via the polypeptide that is coupled to the bead), and resuspending the isolated cell population to produce a cell population that is enriched for polypeptide-responsive cells. For example, if the bead-coupled polypeptide is a Wnt polypeptide, then the cell population will be enriched for Wnt responsive cells. If the bead-coupled polypeptide is a DKK polypeptide, then the cell population will be enriched for DKK responsive cells. All steps are generally performed at room temperature, but can be performed at any temperature that allows binding of cells to the polypeptide-couple beads (e.g., ranging from 4° C. to 37° C., e.g., 4° C., room temperature, 30° C., 37° C., etc.).

The step of isolating the beads can be performed by any convenient method. For example, if the bead is magnetic, the bead-coupled polypeptides (once contacted with a target cell population) can be positioned within the magnetic field of a magnet to produce an isolated cell population comprising cells that are bound to the wnt-coupled magnetic beads. If the beads are not magnetic, techniques such as gentle centrifugation (centrifugation at speeds that do not disrupt cellular integrity) may be used.

After enrichment, if desired, the polypeptide-coupled beads can be removed from the cells by any convenient method (e.g., by contacting the cells with trypsin, e.g., contacting cells with 0.25% trypsin for 10 minutes at 37° C.).

As used herein, the terms “enriching”, “enrichment”, and the like mean an increase in the fraction of target cells that have a desired characteristic (e.g., an increase in the fraction of Wnt-responsive cells of a population, an increase in the fraction of DKK-responsive cells of a population, etc.). A population of cells is considered enriched for the desired cells as long as the fraction of desired cells has increased by greater than 1% (e.g., greater than 2%, greater than 3%, greater than 4%, greater than 5%, greater than 8%, greater than 10%, greater than 12%, greater than 15%, greater than 20%, greater than 30%, greater than 40%, greater than 50%, or greater than 60%). For example, if 80% of the cells of a target cell population are Wnt responsive cells, then an increase to 85% would be considered an enrichment (increased by 5%).

An enrichment can be verified by measuring the fraction of cells that are responsive to the subject bead-coupled polypeptide. For example, the number of Wnt responsive or DKK responsive cells can be determined by any of the means described above for measuring biological activity of a subject Wnt stimulator or inhibitor polypeptide (e.g., count the fraction of cells that have nuclear localized β-catenin, count the fraction of cells that have differentiated after contact with a Wnt stimulator or inhibitor polypeptide, count the fraction of cells that have maintained pluripotency after contact with a Wnt stimulator or inhibitor polypeptide, etc.)

Compositions and Kits

Also provided are compositions and kits for use in the methods. The subject compositions and kits include a biologically active Wnt stimulator or inhibitor polypeptide coupled to a magnetic or glass bead and a buffer that is substantially free of detergent (i.e., a detergent-free buffer). In some embodiments, the bead-coupled polypeptide is at least 70% biologically active when stored in the detergent-free buffer. In some embodiments, the subject compositions and kits include a Wnt polypeptide (e.g., a Wnt stimulator polypeptide, a Wnt3a polypeptide, a Wnt inhibitor polypeptide, a Wnt5 polypeptide, a Wnt11 polypeptide, etc.) coupled to a magnetic bead. In some embodiments, the subject compositions and kits include a Wnt stimulator polypeptide (e.g., a Wnt polypeptide, a Wnt3a polypeptide, a Norrin polypeptide, etc.) coupled to a magnetic bead. In some embodiments, the subject compositions and kits include a Wnt inhibitor polypeptide (e.g., a Wnt polypeptide, a Wnt5 polypeptide, a Wnt11 polypeptide, a DKK polypeptide, an sFRP polypeptide, a Notum polypeptide, a WISE/SOST polypeptide, etc.) coupled to a magnetic bead. In some embodiments, the subject compositions and kits include a Wnt polypeptide (e.g., a Wnt stimulator polypeptide, a Wnt3a polypeptide, a Wnt inhibitor polypeptide, a Wnt5 polypeptide, a Wnt11 polypeptide, etc.) coupled to a glass bead. In some embodiments, the subject compositions and kits include a Wnt stimulator polypeptide (e.g., a Wnt polypeptide, a Wnt3a polypeptide, a Norrin polypeptide, etc.) coupled to a glass bead. In some embodiments, the subject compositions and kits include a Wnt inhibitor polypeptide (e.g., a Wnt polypeptide, a Wnt5 polypeptide, a Wnt11 polypeptide, a DKK polypeptide, an sFRP polypeptide, a Notum polypeptide, a WISE/SOST polypeptide, etc.) coupled to a glass bead. In some embodiments, the compositions and kits may further include at least one additional bead-coupled Wnt stimulator or inhibitor polypeptide. As such, the subject compositions and kits include two or more bead-coupled Wnt stimulator or Wnt inhibitor polypeptides.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

For further elaboration of general techniques useful in the practice of this invention, the practitioner can refer to standard textbooks and reviews in cell biology, stem cell biology, tissue culture, embryology, and developmental biology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. In the following description, reference will be made to various methodologies known to those of skill in the art of immunology, cell biology, and molecular biology. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

### Example 1

### Materials and Methods

Production and immobilization of Wnt proteins. Recombinant mouse Wnt3a or Wnt5a proteins were produced in Drosophila S2 cells grown in suspension culture, and purified by Blue Sepharose affinity and gel filtration chromatography as described (Willert et al., Nature. 2003 May 22;423(6938):448-52: incorporated herein by reference). Wnt3a activity was determined in a luciferase reporter assay using L cells stably transfected with the SuperTOPFlash reporter as described (Mikels and Nusse, PLoS Biol. 2006 Apr;4(4):e115: incorporated herein by reference). Wnt3a or Wnt5a were immobilized onto 2.8 μm Dynabeads® M-270 Carboxylic Acid (Invitrogen). The carboxylic acid groups were activated at room temperature for 30 min by incubating 30 μg of beads with 50 μl carbodiimide (from a 50 mg/ml stock solution, dissolved in 25 mM cold 2-(N-morpholino)ethanesulfonic acid (MES) buffer, pH 5) and 50 μl N-hydroxyl succinimide (from a 50 mg/ml stock solution, dissolved in MES buffer, pH 5). After activation the beads (about 30 μg) were washed three times with 100 μl of 25 mM MES buffer pH 5.

Coupling the Wnt protein to the activated beads according to the manufacturer's instructions resulted in inefficient coupling. Instead, to achieve Wnt immobilization, 100 μl of 20 ng/μl purified Wnt protein (total of about 2 μg of protein) was concentrated by centrifugation for 30 minutes at 4° C. using a Centricon® filter. About 10-15 it were recovered, resulting in a purified Wnt protein at a concentration of about 130-200 ng/μl. The entire recovered amount of Wnt protein was mixed with the activated beads (about 30 μg beads in 100 μl MES buffer) for 1-2 minutes at RT (Longer incubations resulted in decreased coupling efficiency). The mixture was then diluted 1:5 in cold 25 mM MES buffer, pH 5 and incubated at room temperature with gentle rocking for one hour. The Wnt-coupled beads were then washed three times with MES pH 5 and an additional three times with phosphate buffered saline (PBS) pH 7.4. The Wnt beads were stored in PBS/1% BSA buffer at 4° C.

To generate inactive variants of immobilized Wnt3a, Wnt3a protein was immobilized onto beads followed by incubation with varying concentrations (1-10 mM) Dithiothreitol (DTT) for 12 min at 37° C. The Wnt beads were then washed six times with PBS and stored in PBS/1% BSA buffer at 4° C. The activity of the DTT treated Wnt beads was inversely proportional to the concentration of DTT.

Purified R-spondin-1 and DKK-1 proteins were purchased from R&D systems. 6 μg purified R-spondin-1 was covalently immobilized onto 2.8 μm Dynabeads® M-270 Carboxylic Acid (Invitrogen) according to manufacturer instructions. To immobilize DKK-1, 10 μg aldehyde coated glass beads (5 micron, Xenopore) were washed three times with PBS and then incubated with 4 μg purified DKK1 for one hour. The DKK-1 beads were washed with PBS three times and stored in PBS/1% BSA buffer at 4° C.

Wnt5a activity was determined by inhibiting Wnt3a activity in a luciferase reporter assay using 293 cells stably transfected with the SuperTOPFlash reporter as described (Mikels and Nusse, PLoS Biol. 2006 Apr;4(4):e115).

Cell lines and Cell culture: Mouse Embryonic Stem (ES) cells (ESCs) were cultured in ES cell medium (DMEM plus 15% fetal bovine serum (Hyclone), 1 mM sodium pyruvate, MEM non-essential amino acids, 50 μM 2-mercaptoethanol, 100 U/ml penicillin, 100 μg/ml streptomycin (all from Invitrogen) and 1,000 U/ml LIF (Chemicon)) supplemented with 100 ng/ml Wnt3a protein on gelatin-coated plates. After the second passage, ES cells were cultured over night and then switched to N2B27 medium consisting of one volume of DMEM/F12 combined with one volume of Neurobasal medium supplemented with 0.5% N2 Supplement, 1% B27 Supplement, 0.033% BSA 7.5% solution, 50 μM 2-mercaptoethanol, 2 mM Glutamax, 100 U/ml penicillin and 100 μg/ml streptomycin (all from Invitrogen) with 100 ng/ml Wnt3a protein. Media and recombinant proteins were changed daily in all experiments except where indicated otherwise.

After 3-5 days, the cells formed colonies and were ready to be passaged. For time-lapse imaging of single cells, the colonies were washed twice with DPBS and then incubated with cell dissociation buffer (Gibco) at 37° C. for 5 minutes followed by gentle re-suspension until a single cell suspension was achieved as determined by light microscopy. The cells were pelleted and resuspended in N2B27 medium medium or N2B27 medium supplemented with MEK inhibitor PD0325901 (1 μM) and GSK3 inhibtor CH99021 (3 μM) together known as 2i (8). mESC Cell lines: Sox2-GFP mESCs (23) a gift from Dr. Konrad Hochedlinger, Stella-GFP mESCs (20) a gift from Dr. Azim Surani, OCRG9 mESCs (21) a gift from Dr. Hitoshi Niwa, H2B-Venus ES cells a gift from Dr. Timm Schroeder. Female LF2 ES cells were obtained from Dr. Joanna Wysocka, Stanford.

Plasmids and mESC transfection: pEGFP-CENT1 was obtained from Dr. Song Hai Shi, the mouse ninein (GenBank/EMBL/DDBJ accession number AY515727) plasmid was provided by Dr. Michel Bornens, XE241 Rat frizzled-1-GFP-CS2P+ was purchased from Addgene (Plasmid 16817). Transient transfections were performed with X-tremeGene HP-DNA transfection reagent on mESCs according to the manufacturer's instructions (Roche). At 24 h post-transfection, cells were plated into a 6 well plate, allowed to recover for 6-8 hours and then prepared for time-lapse imaging experiments.

Marker staining and immunohistochemistry: For immunostaining, chambered coverglasses were coated with 15 mg/ml human plasma fibronectin (Sigma). 5000 cells/well were cultured with 2.25 μg Wnt beads in N2B27 medium. After 16 hours the cells were washed with DPBS and fixed with 4% paraformaldehyde for 30 min at 4° C., washed thrice with Staining buffer (0.1% BSA, 0.001% Tween 20, and 0.05% sodium azide in PBS), and blocked with 10% normal donkey serum (NDS)/Staining buffer for 30 min. Samples were then incubated with 1:200 primary antibody in Staining buffer overnight at 4° C., washed three times with Staining buffer and detected with 1:600 dilution of secondary antibodies (Alexa fluor 594, Alexa fluor 647) followed by confocal imaging. The primary antibodies used were: APC (Santa Cruz, sc 7930), anti-H3K27me3 (active motif 39158), anti-Claudin6 (Santa Cruz, sc17669), anti-Stella (abcam, ab19878), anti-Rex1 (abcam, ab28141), anti β-catenin (BD 610154) and anti-Lrp6(T1479) a gift from Dr. Gary Davidson.

Confocal imaging. The Zeiss Meta LSM510 confocal was utilized and a series of Z stack images were collected with either 63x/NA 1.4 oil or 40x/NA 1.3 oil objective. The Z series were analyzed by Volocity software (Perkin Elmer). Only a fine filter was applied to reduce the noise in the image. A three dimensional reconstruction was performed and a snapshot was made and represented in JPEG image format. Some of the panels contain XYZ views.

Two-dimensional time-lapse imaging: Cell preparation: Single cell suspension of 5000 reporter ES cells (in N2B27 media) were mixed with 2.25 μg Wnt beads and co-cultured in 8 well chambered coverglass slides. The cells and beads were incubated for 1 h at 37° C. and 5% CO2 before starting the time-lapse experiment.

Time-lapse microscopy for all experiments was performed using a DMI6000 B system (Leica) at 37° C. and 5% CO2. Bright field and fluorescence images were acquired every 20 to 30 min using 20x/NA 0.7 or 40x/NA 0.75 objective, a Cascade II camera (1024×1024), a 300 W Xenon lamp and Metamorph acquisition software (Molecular Devices). Metamorph software or Image J software were used for image analysis and for the generation of movies (QuickTime and AVI). In some of the analysis, a Gaussian blur filter (2×2) was used to reduce the noise in fluorescence images and the image contrast was manually enhanced for optimal recognition of the relevant cellular features separately for each wavelength. All individual frames of time-lapse acquisition are displayed as JPEG image format. All cell tracking was done manually; the presented analysis does not rely on data generated by an unsupervised computer algorithm for automated tracking. Only single cells contacting one bead or more that could be identified clearly were used for analysis, and all cells touching other cells or with questionable identity were excluded from the analysis.

Signal intensity quantification of Nanog-Venus: for each frame, the cells were recognized using both the contrast of the bright-field image and the fluorescent signals from the Nanog-Venus image. The Nanog-Venus image was then processed by local background subtraction. The mean and standard deviation of the Nanog signal intensity from each cell are determined by all the pixels within the mask, excluding the pixels of the 50 highest and the 50 lowest intensities. Student's-t test was used to examine the difference between the cell with bead(s) and the one without bead(s). All above processes were performed using Matlab 7.11.0 (Mathwork.)

Bessel beam plane illumination microscopy. Mouse ES cells expressing H2B-Venus protein were mounted onto 18 mm coverslips at a 45° angle in the y-z plane defined by the axes of excitation and detection objectives, and translated with sample stages to place the desired part of the specimen in the imaging volume. The sample chamber was filled with N2B27 media. For time-lapse movies, approximately 150-250 two-dimensional images comprising a 40 μm thick three-dimensional volume were captured every 30-60 seconds with 488 nm excitation. Point spread functions were calculated, and images were deconvolved as previously described. Three-dimensional renderings were created using Amira software (Visage Imaging) or Volocity (PerkinElmer). The voxel size of an image is 133 nm, 133 nm, 150 nm in x, y, z planes respectively.

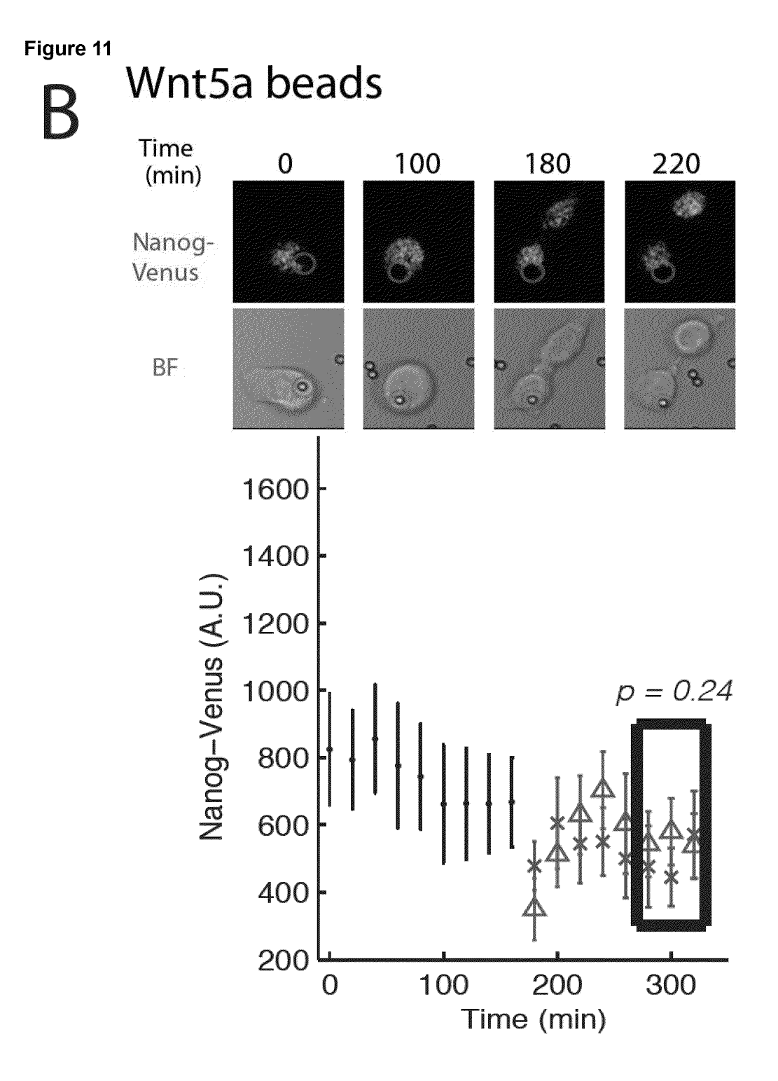

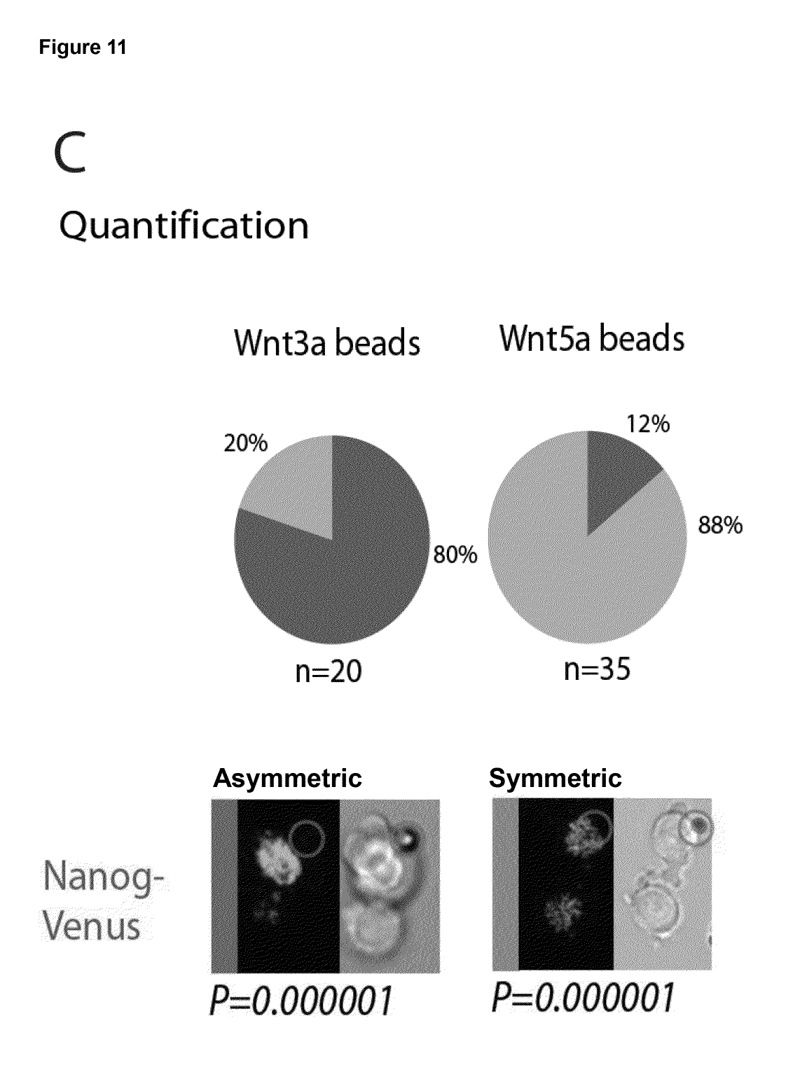

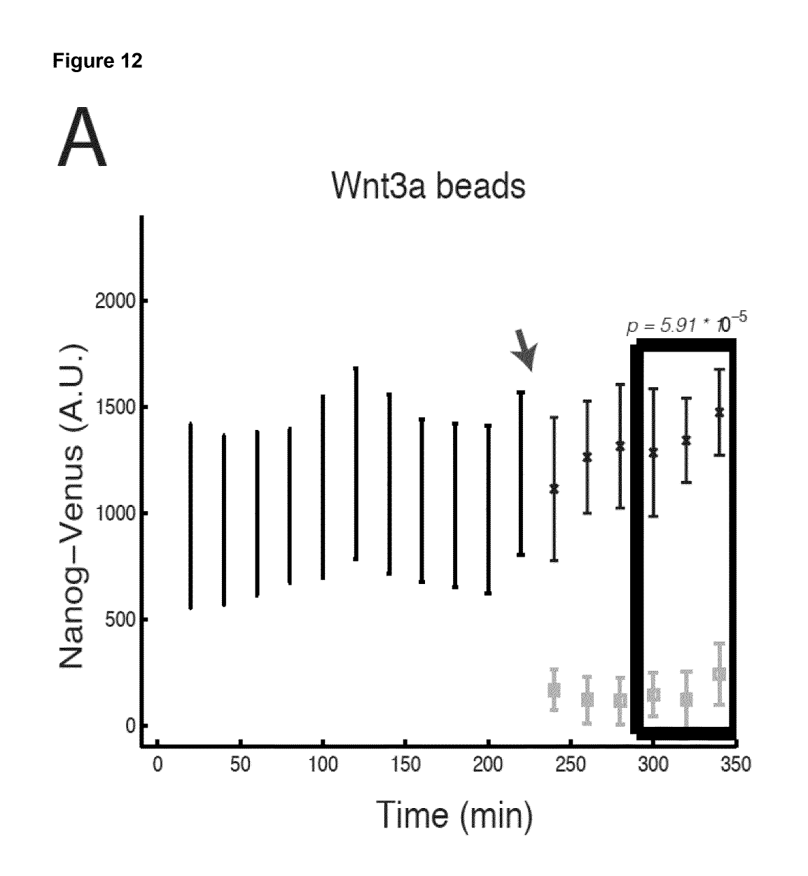

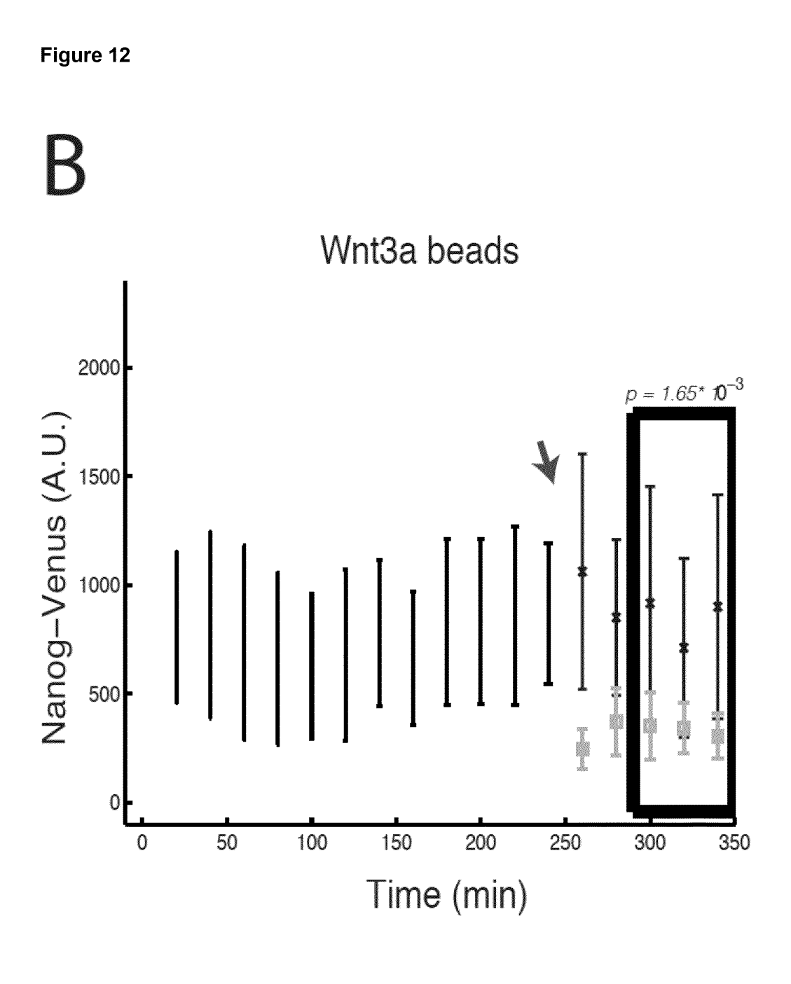

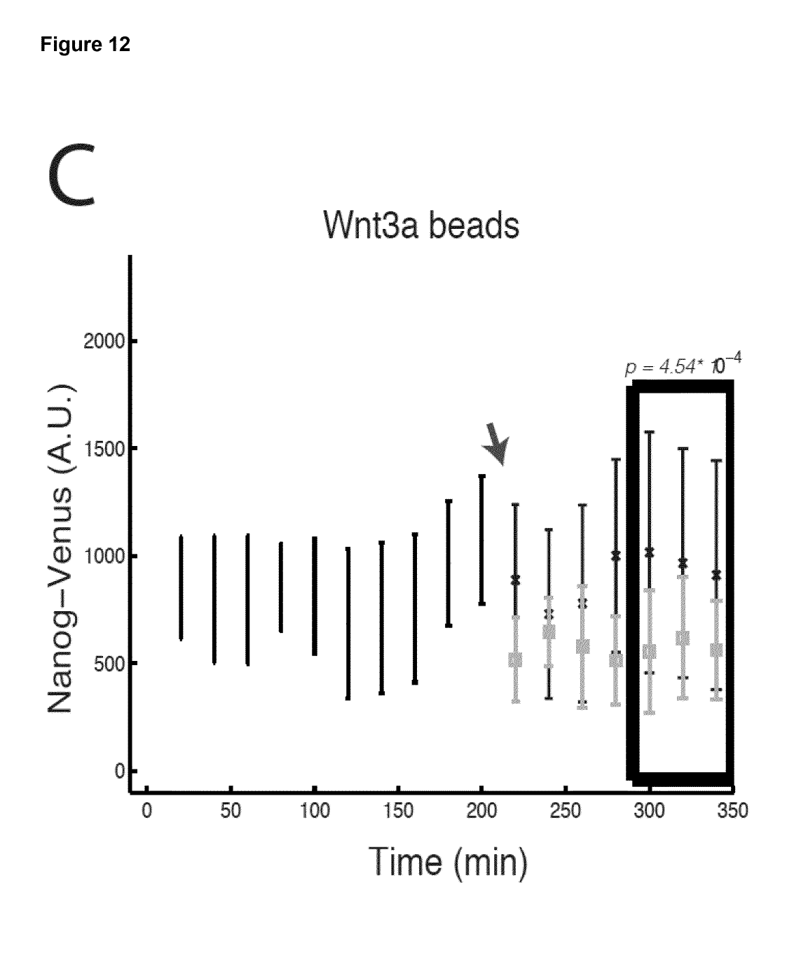

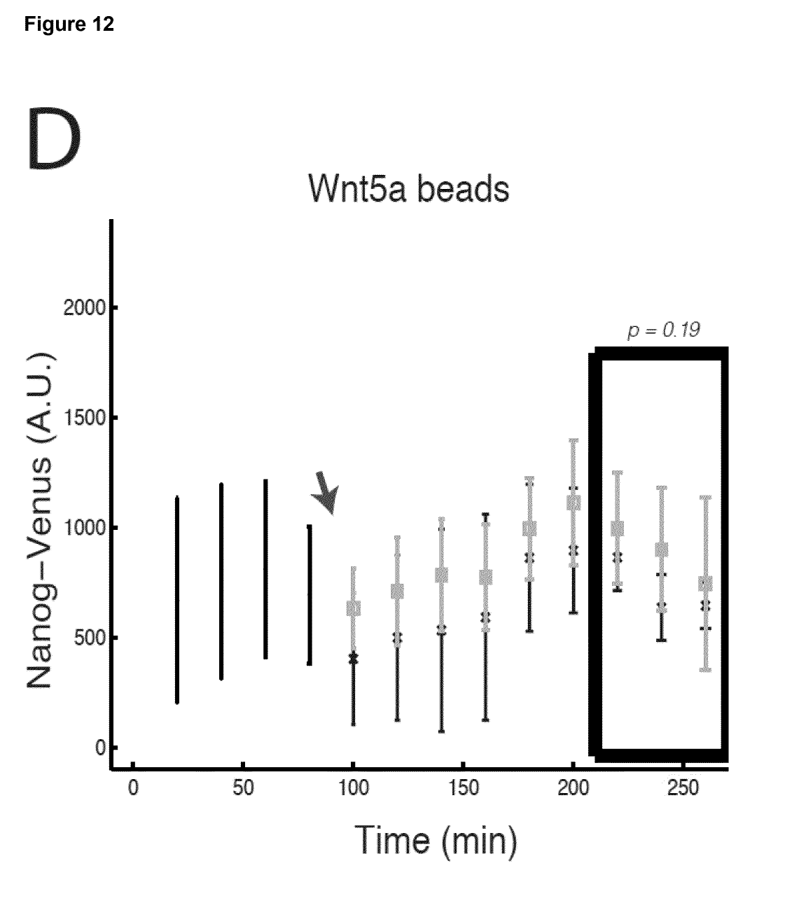

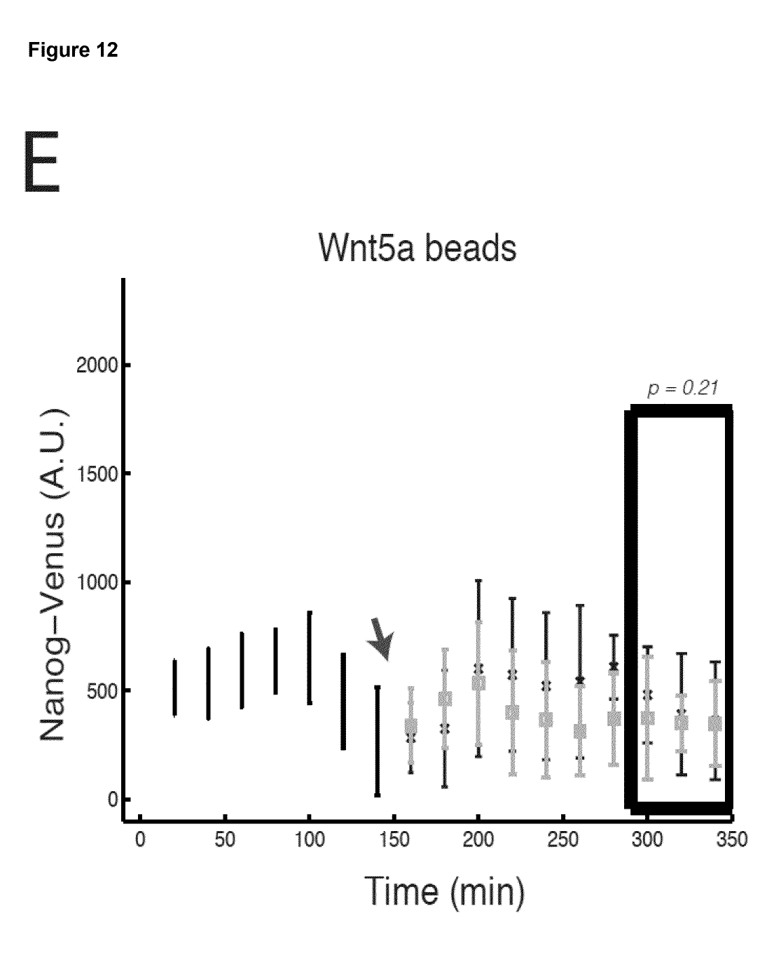

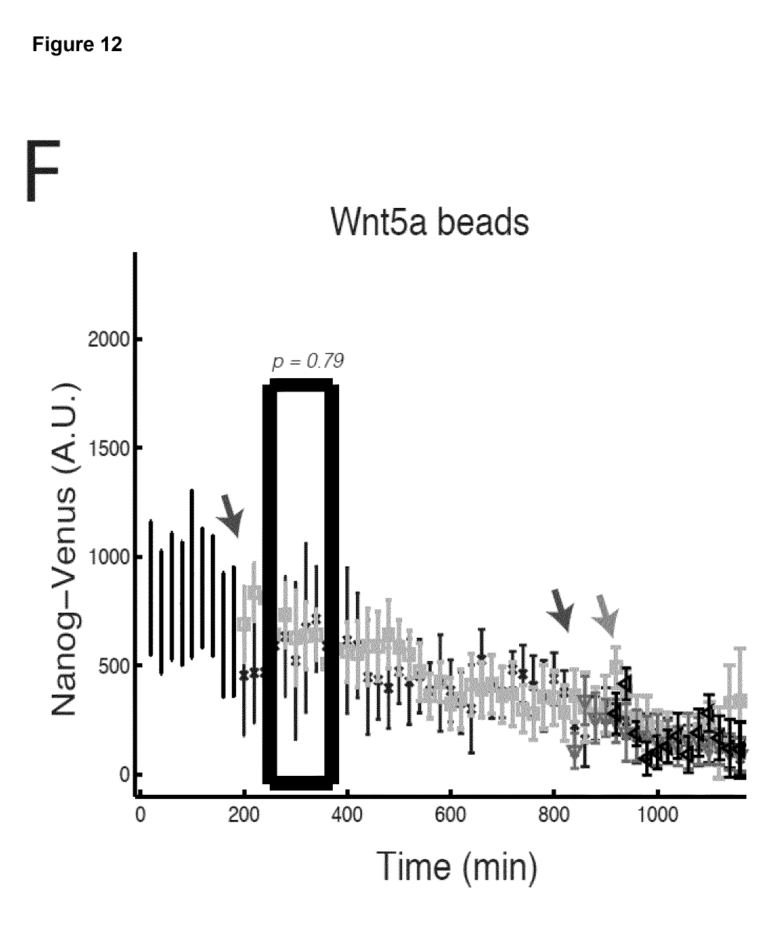

Statistics: All P-values were calculated according to Fisher exact probability test except those in FIGS. 11 and 12. The P-values are calculated for the same category in Wnt3a versus Wnt5a bead treatments except where indicated otherwise.

### Results

To ask whether directional Wnt signals influence asymmetric stem cell division, we immobilized Wnt proteins on beads, applied the beads to embryonic stem (ES) cells, and followed single cells with live imaging. At the single cell level, a Wnt-bead acts as a positional cue and induces asymmetry of Wnt/β-catenin signaling components, orients the plane of mitotic division, and directs asymmetric inheritance of centrosomes. By time-lapse microscopy, we found that the Wnt-beads led to asymmetric inheritance of centrosomes and orientation of the mitotic spindle. Before cytokinesis was completed, the Wnt-proximal daughter cell expressed high levels of nuclear β-catenin and pluripotency genes, whereas the distal daughter cell acquired hallmarks of differentiation. Blocking Wnt signaling locally produced asymmetric cell fates in the opposite manner. Thus, a spatially restricted Wnt signal induces an oriented cell division that generates distinct cell fates at predictable positions relative to the Wnt source.

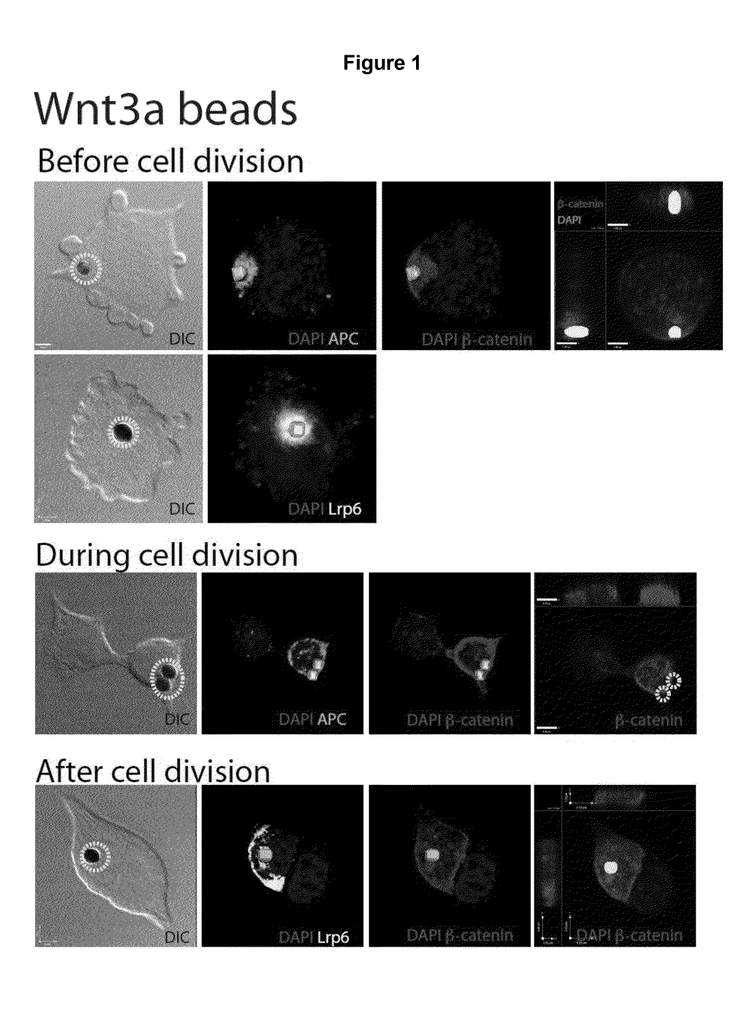

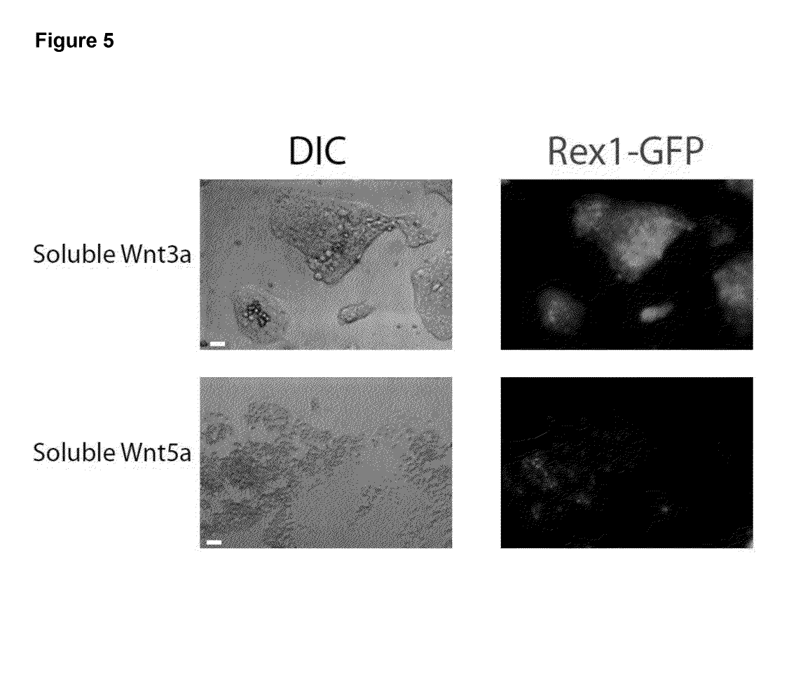

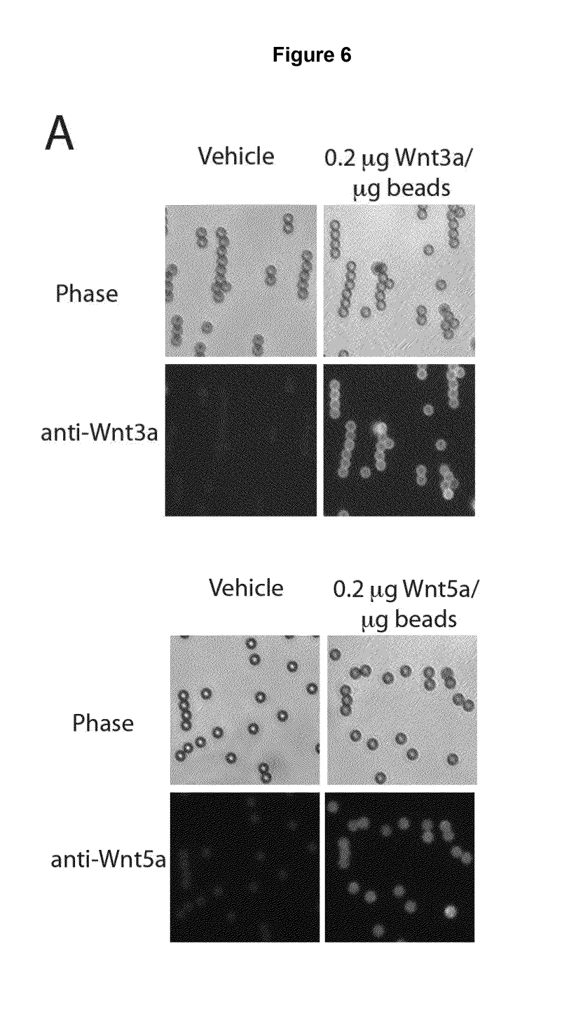

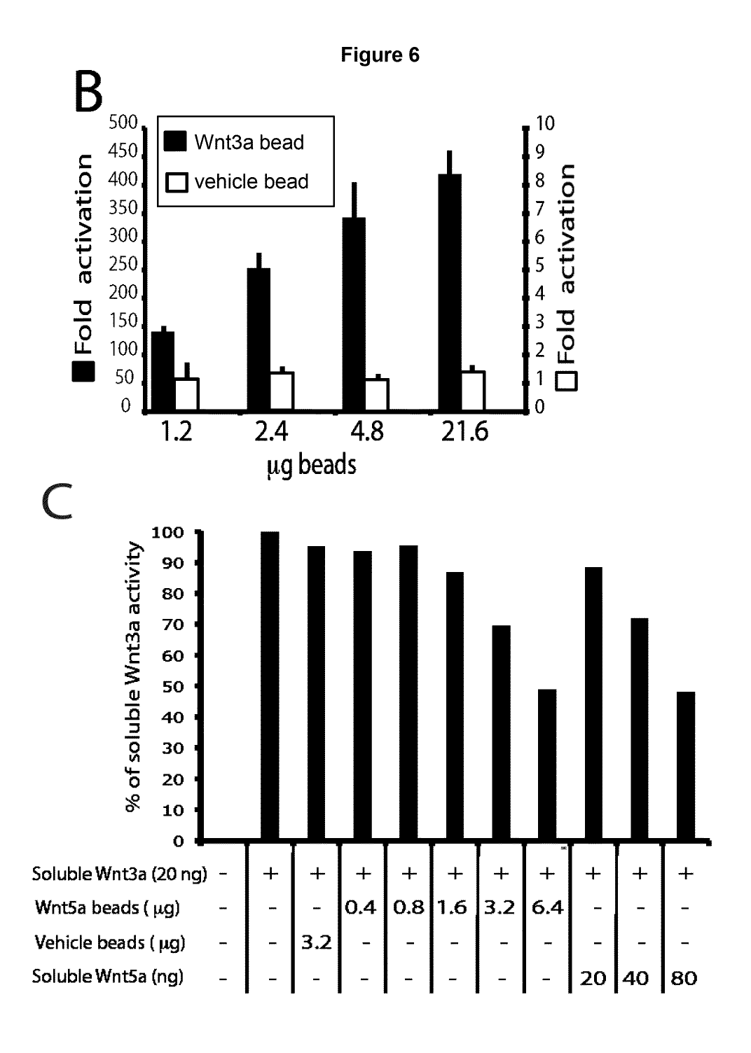

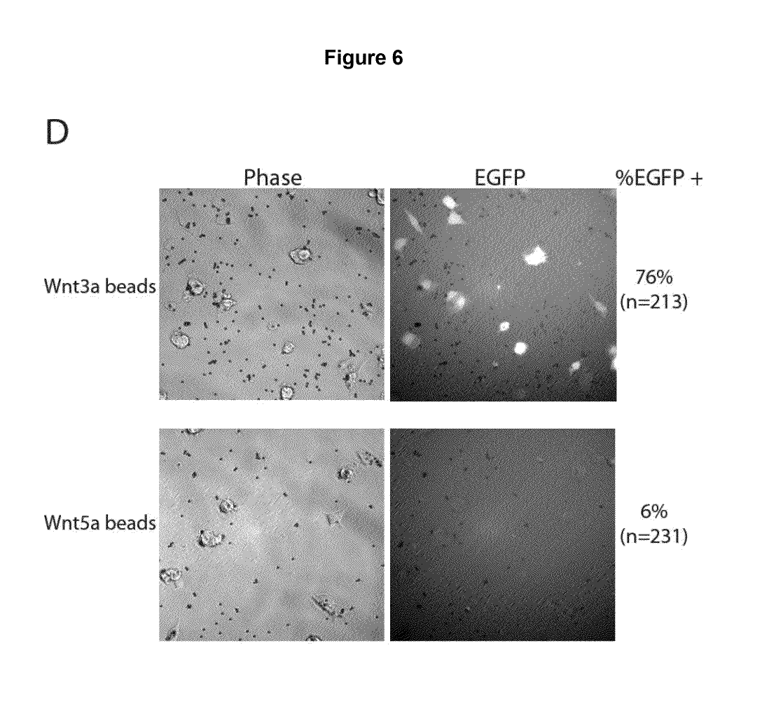

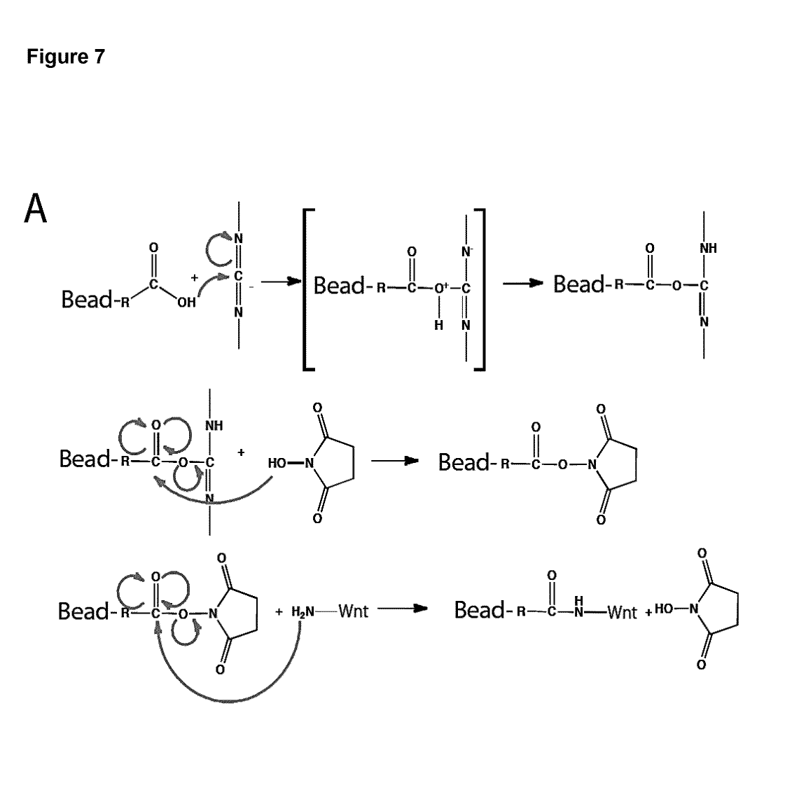

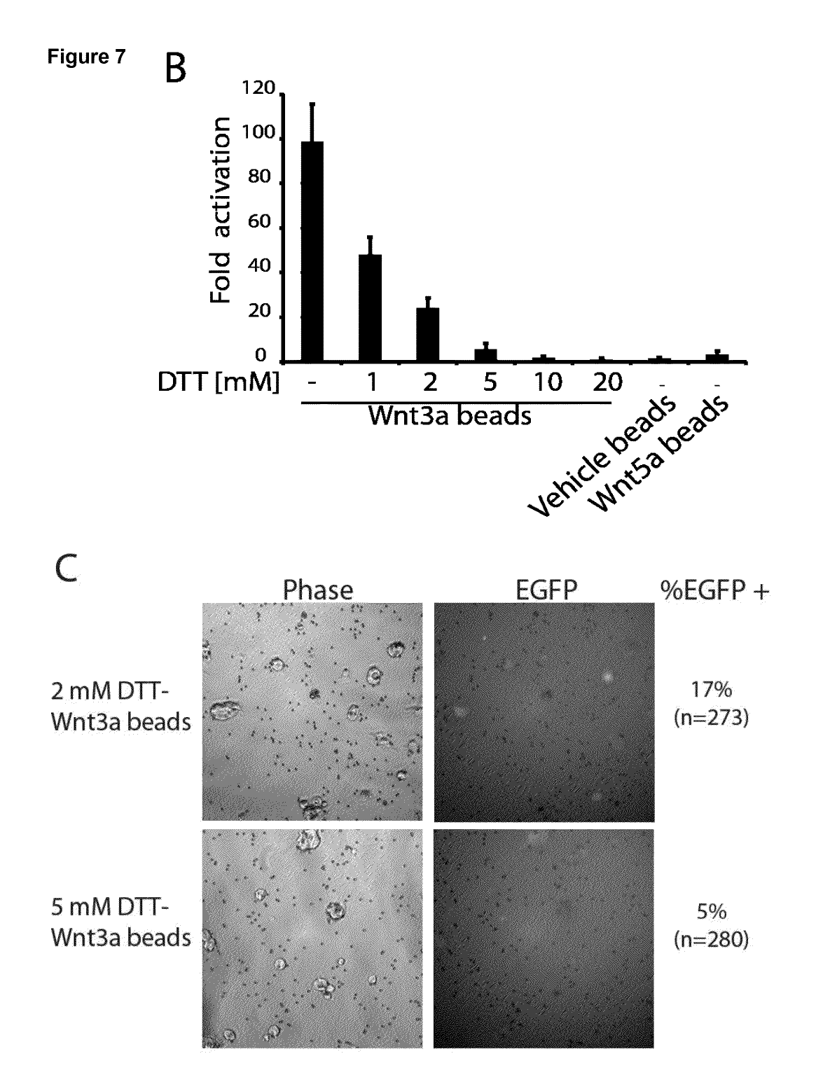

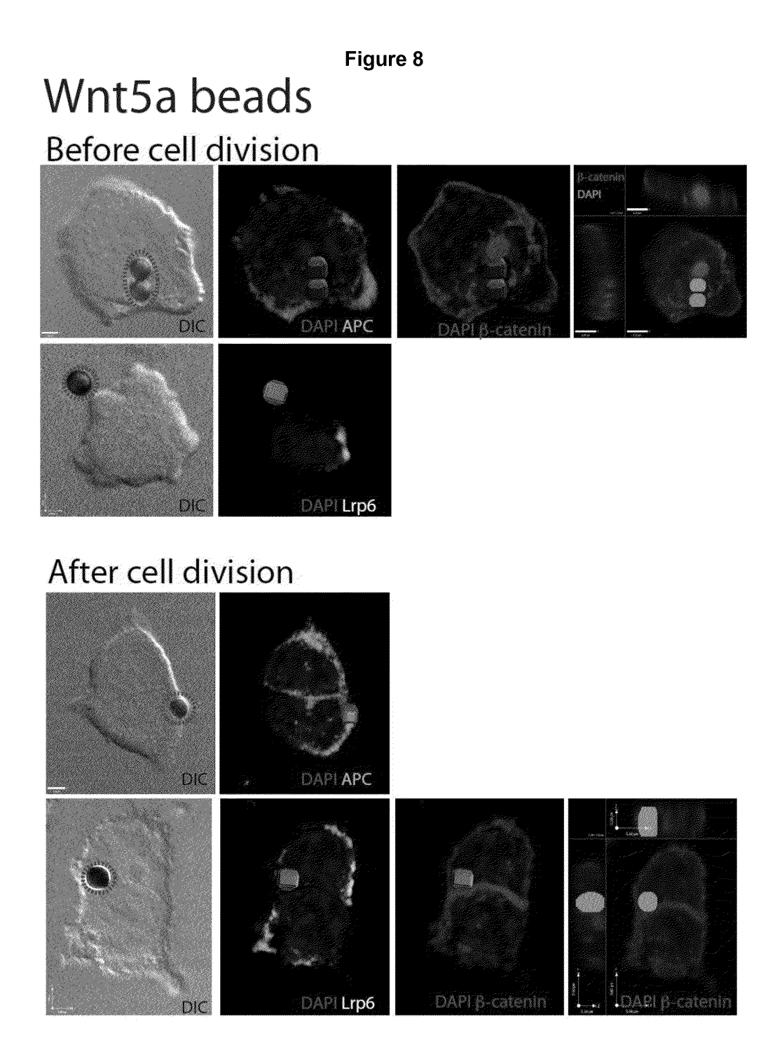

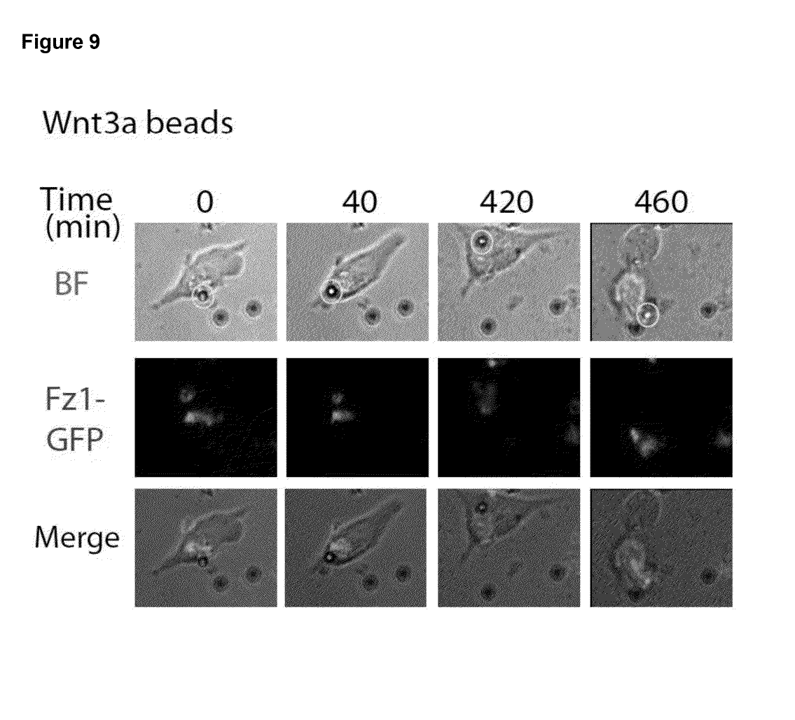

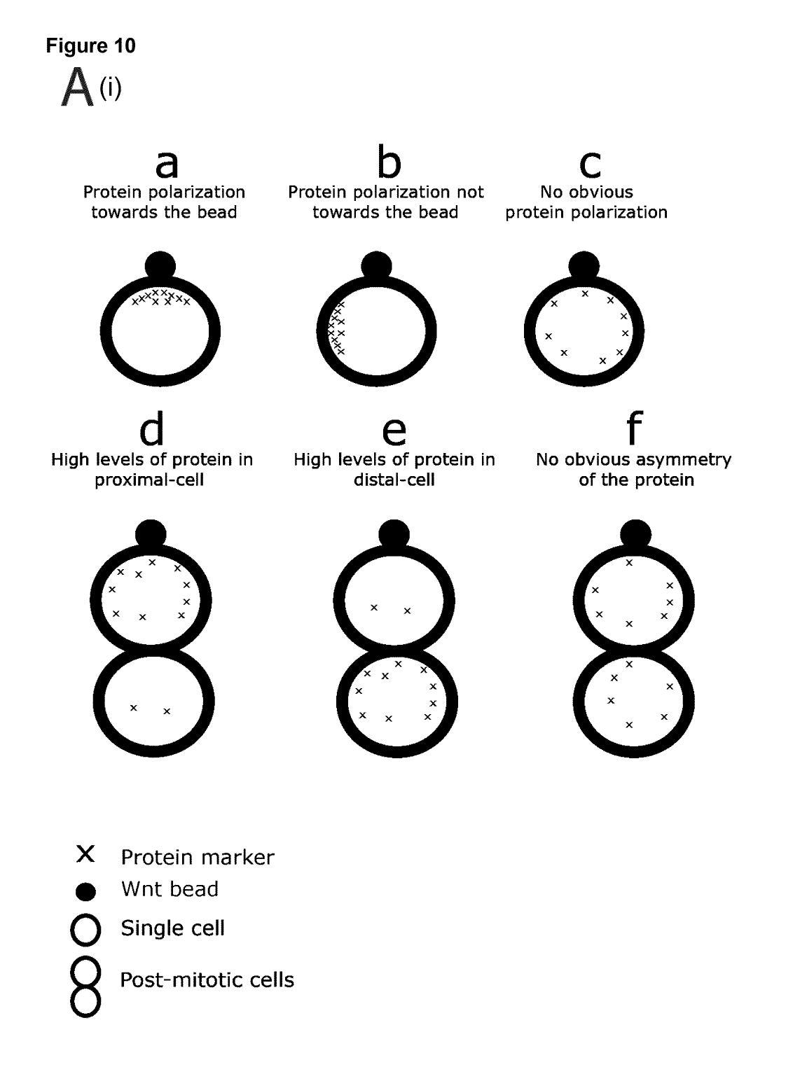

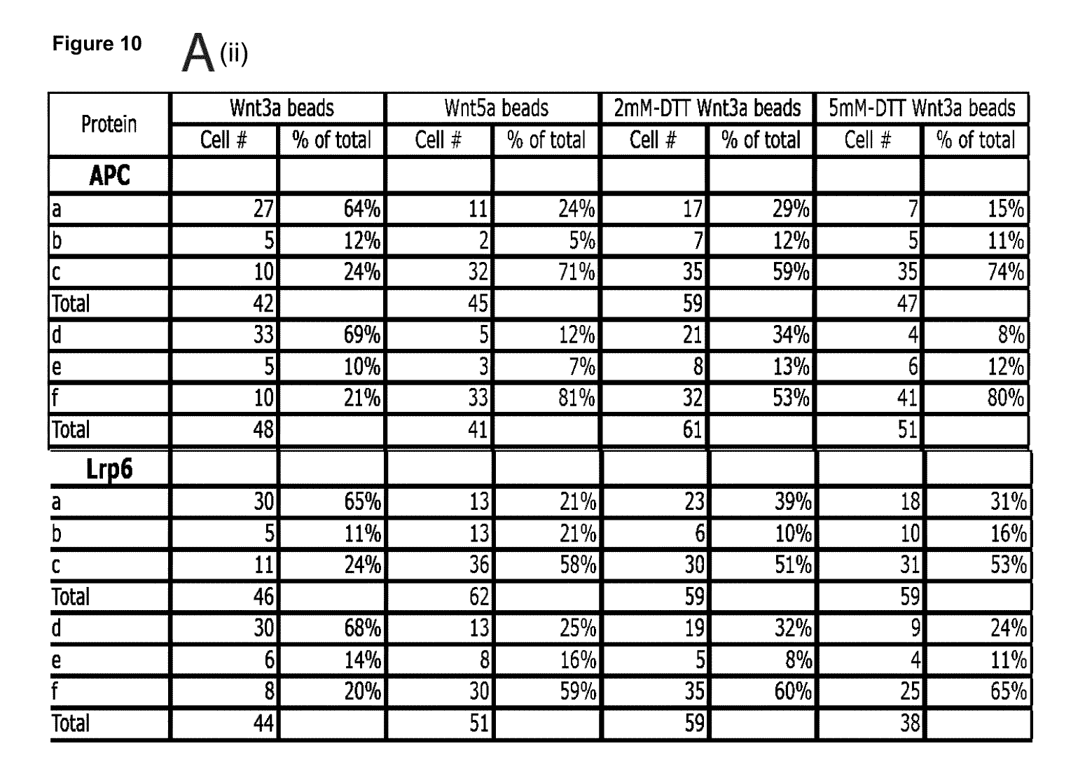

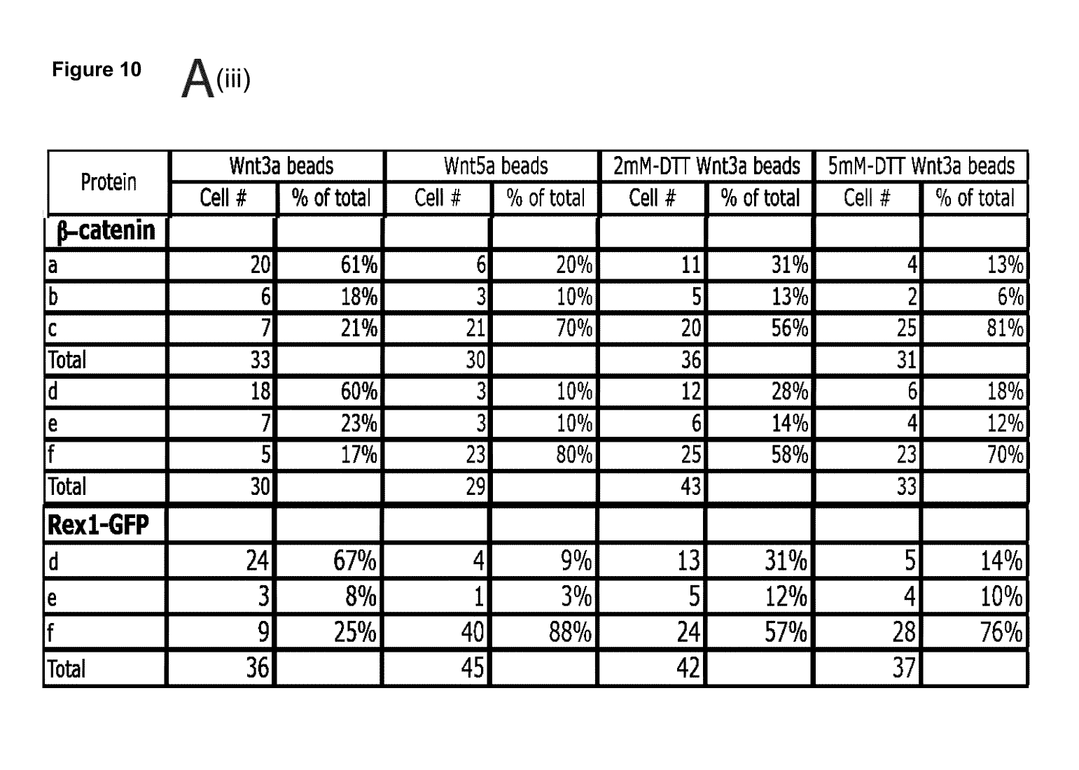

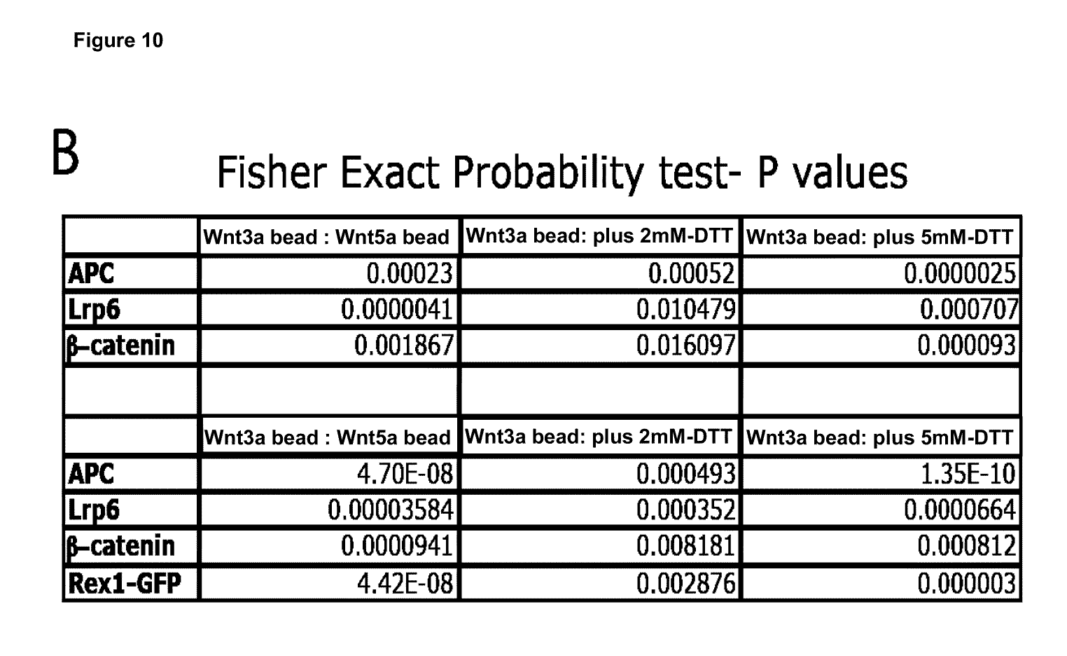

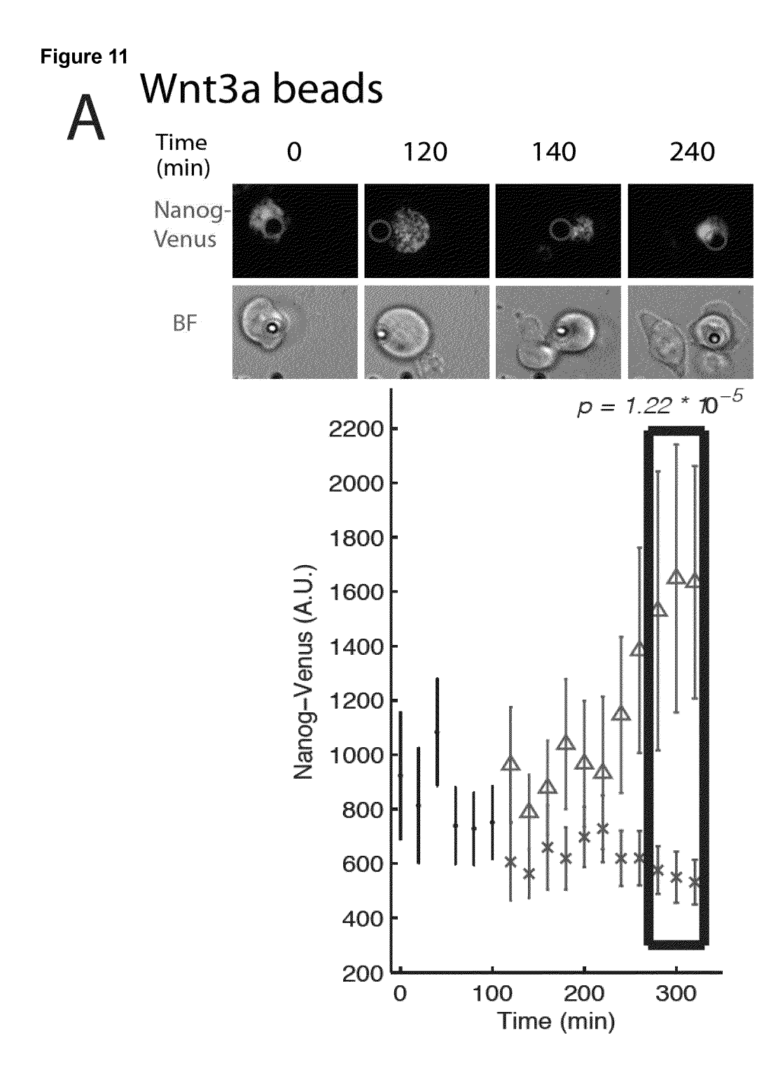

We determined the consequences of presenting a spatially localized Wnt signal to ES cells, by taking a bioengineering approach in the form of Wnt signals bound to beads and following single cells with live imaging. While Wnt3a maintains ES cell pluripotency, the Wnt5a protein, which commonly operates through a non-β-catenin dependent pathway did not (FIG. 5 and FIG. 12F), allowing us to use Wnt5a as a control. To generate a spatially localized source of Wnt signals, we immobilized purified Wnt3a or Wnt5a proteins chemically to beads (FIG. 6A and 7A) and confirmed their biological activity (FIG. 6B-D and FIG. 7B and C). ES cells were plated at low density in the presence of LIF, and individual cells with a bead attached were followed by live cell microscopy as they divided. We examined the location of Wnt signaling components by antibody staining. In the presence of Wnt3a beads (FIG. 1), but rarely with Wnt5a beads (FIG. 8), the Wnt receptor LRP6 became asymmetrically localized to the side of the ES cell contacting the bead. Moreover, a Frizzled1-GFP fusion protein (FIG. 9) and the Adenomatous Polyposis Coli (APC) protein, a component of the β-catenin destruction complex, were detected in close proximity to the Wnt3a beads (FIG. 1 and FIG. 10). The asymmetric distribution of these Wnt components was maintained after the cells had divided: the daughter cell in proximity to the Wnt3a bead retained high levels of LRP6 and APC, whereas the distal daughter cell had much lower levels of these proteins (FIG. 1 and FIG. 10).

In pre-divisional ES cells contacting Wnt3a beads, β-catenin was distributed asymmetrically close to the bead, overlapping with the location of APC (FIG. 1 and FIG. 10). During division, β-catenin was retained at high levels in the prospective proximal daughter cell, both at the cell membrane and in the nucleus. When cytokinesis was complete, this asymmetric β-catenin staining pattern was maintained, with high levels in the Wnt3a proximal cell, in particular in the nucleus (FIG. 1).

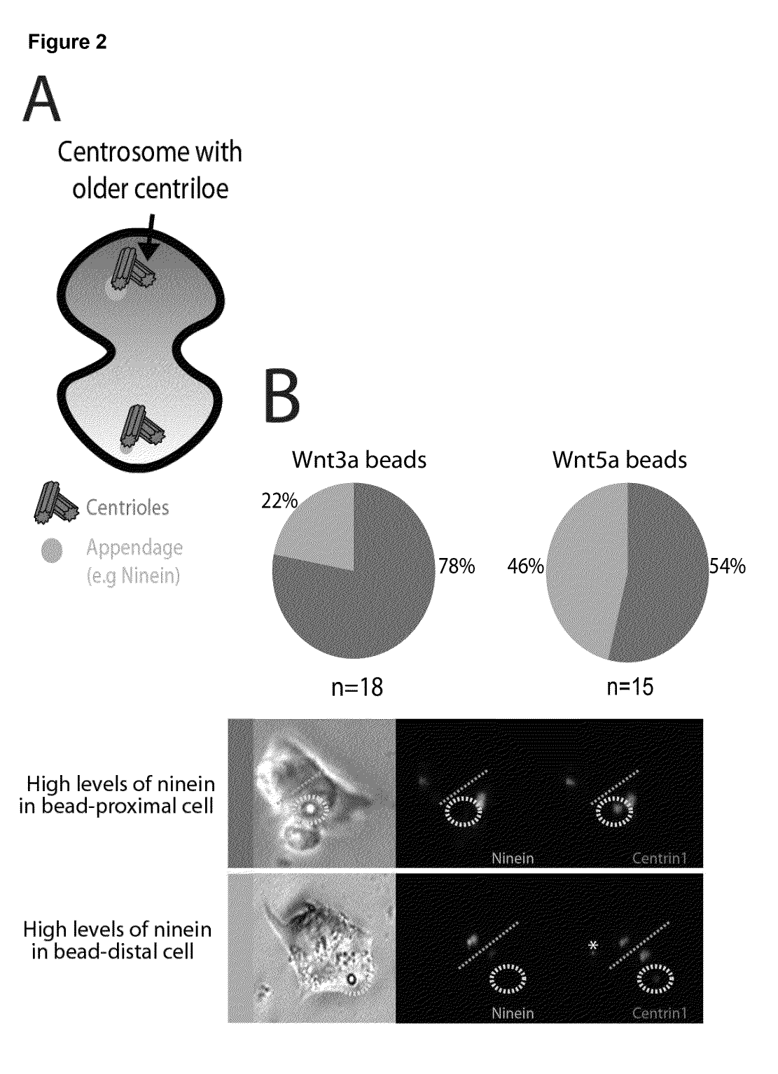

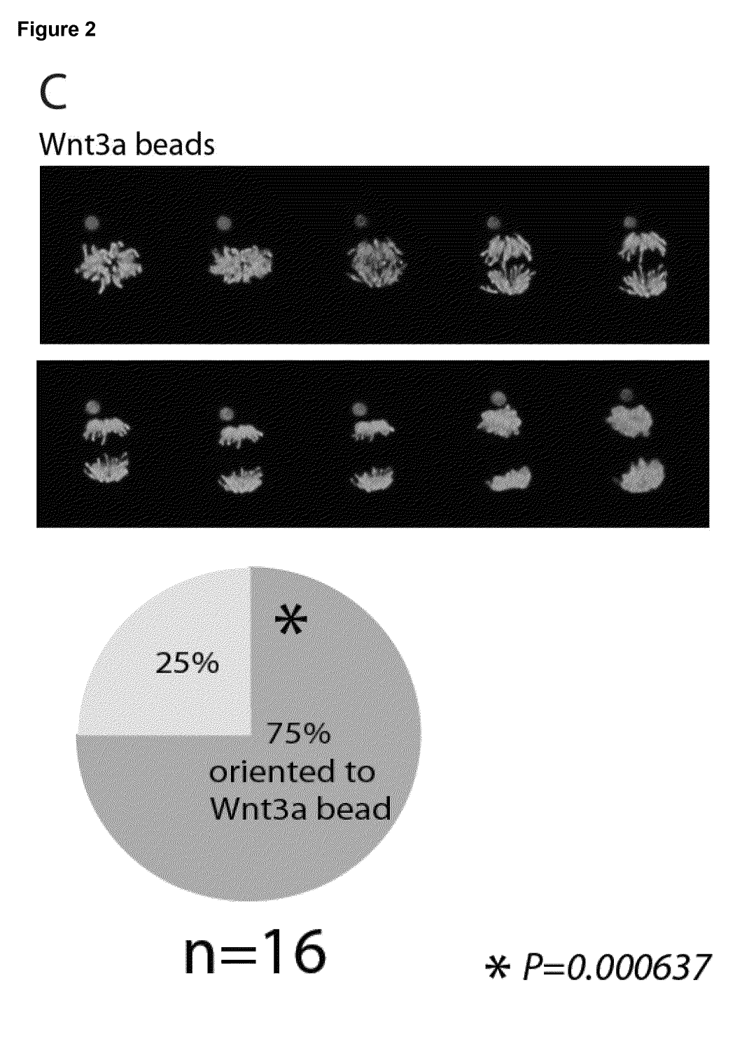

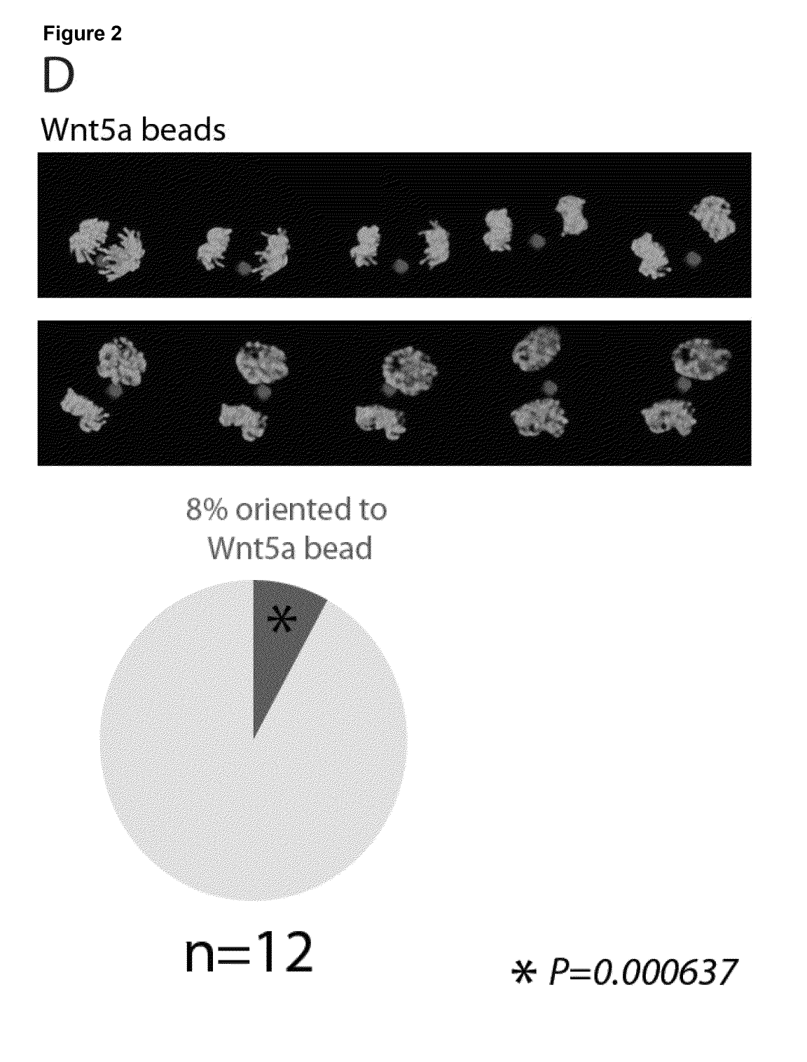

Wnt pathway components can interact with astral microtubules and other components of the mitotic spindle, including centrosomes. We investigated the effect of Wnt3a and Wnt5a beads on the asymmetric inheritance of the centrosomes by expressing tagged Centrin1 (a component of the centriole) and the appendage component Ninein. Ninein marks the centrosome with the older centriole, whereas the other centrosome receives new centrioles that initially lack these structures (FIG. 2A). By the end of division, centrosomes in 78% of the cells (n=18) that were attached to Wnt3a beads had high levels of Ninein (FIG. 2B). In contrast, the segregation of Ninein was almost random in the presence of the Wnt5a beads (FIG. 2B). Thus, Wnt3a beads specify the asymmetric inheritance of centrosomes.

Since centrosomes orient the mitotic spindle, we investigated whether Wnt beads direct the orientation of cell division and partitioning of chromosomes during mitosis. ES cells expressing a Histone 2B-Venus chimeric protein to mark chromosomes (FIG. 2C and D) were incubated with Wnt beads and monitored during mitosis by rapid three-dimensional imaging of living cells. In 75% of the dividing cells (n=16), the axis of mitotic division was oriented in line with the Wnt3a bead (FIG. 2C) whereas only 12% of divisions were oriented toward Wnt5a control beads (n=12; FIG. 2D).

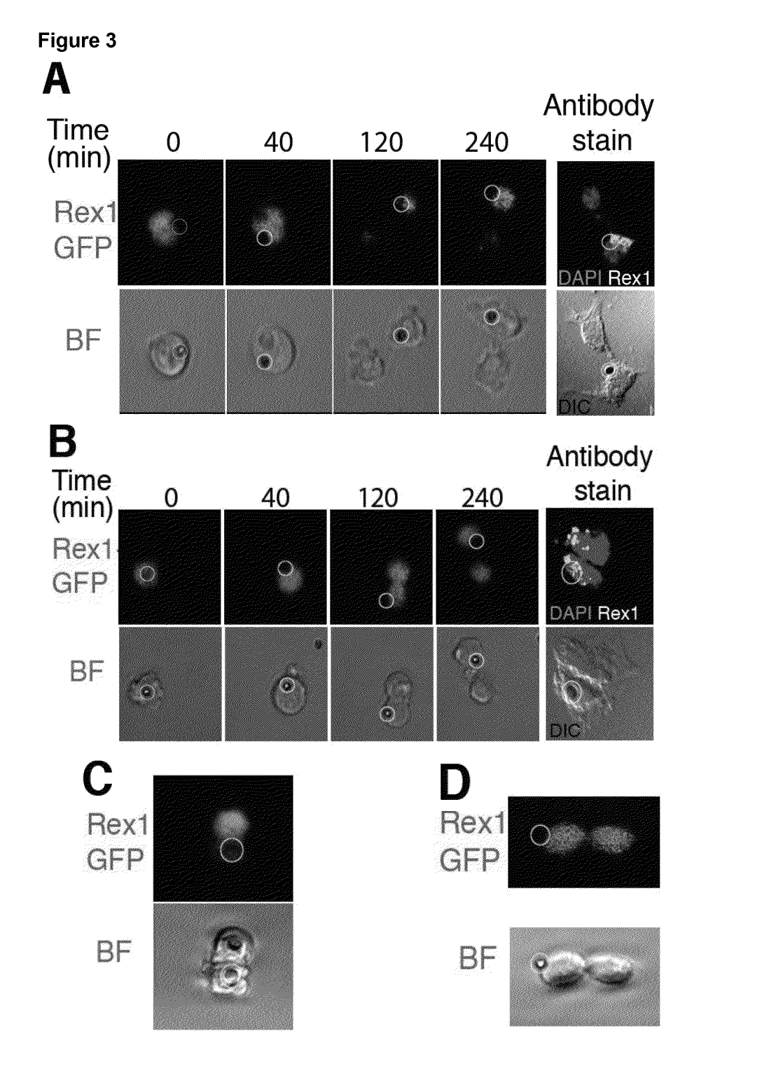

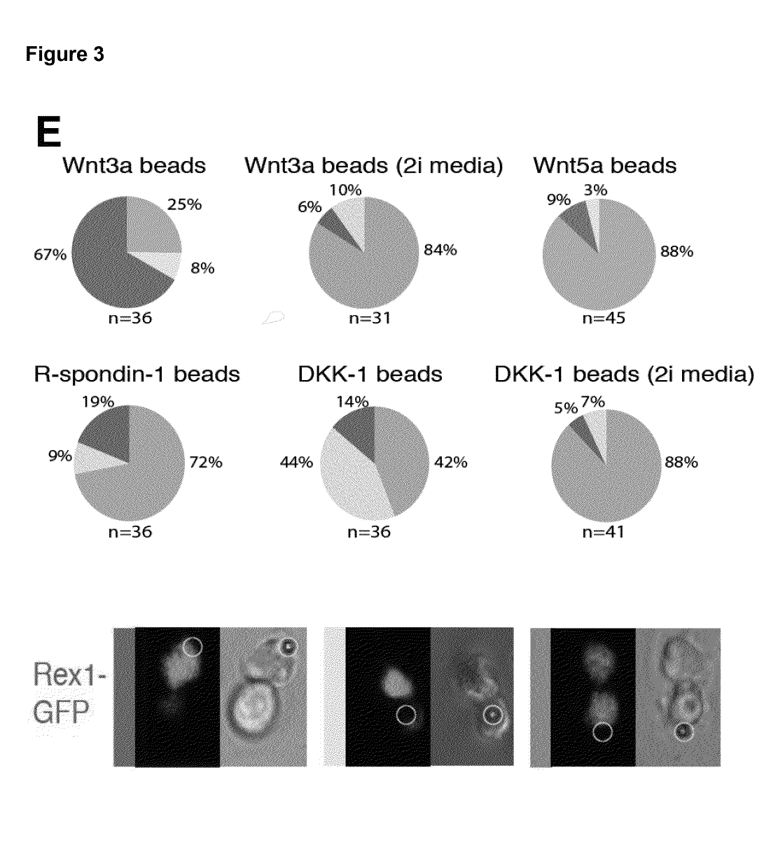

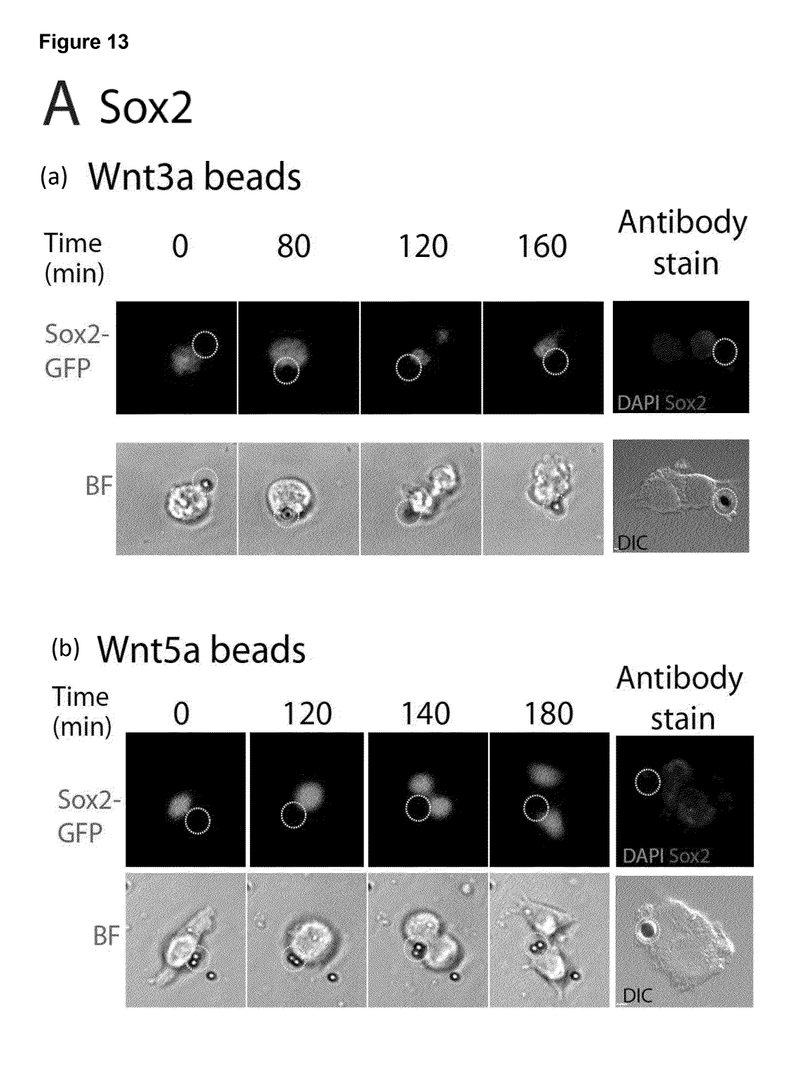

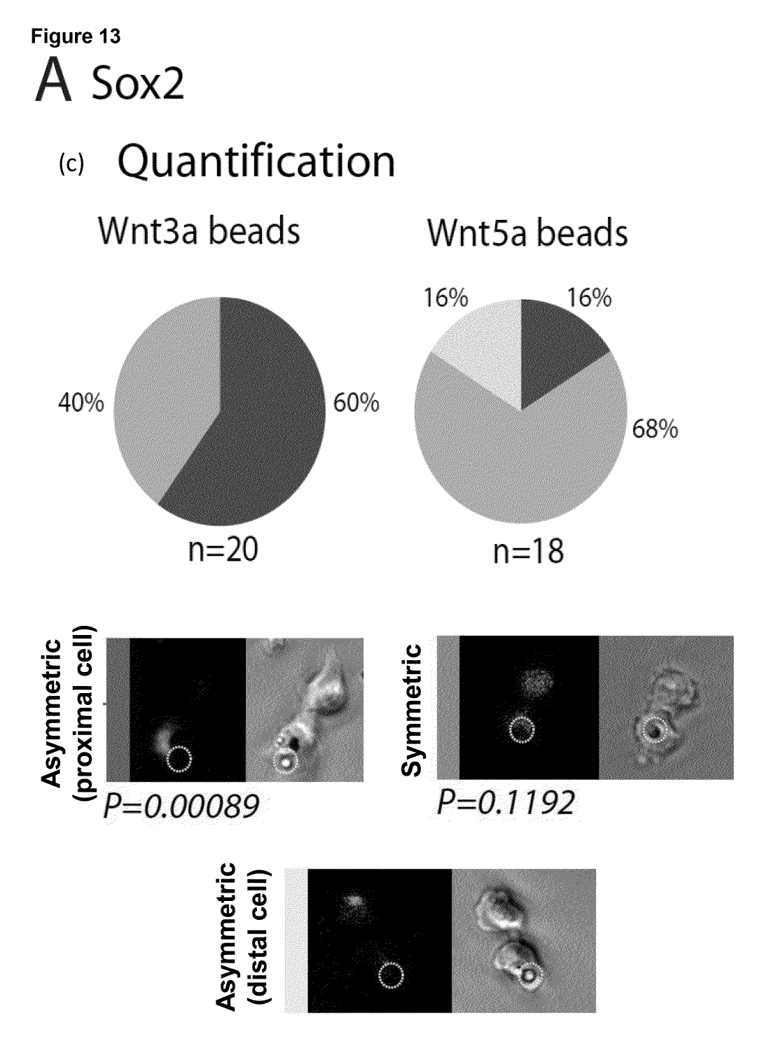

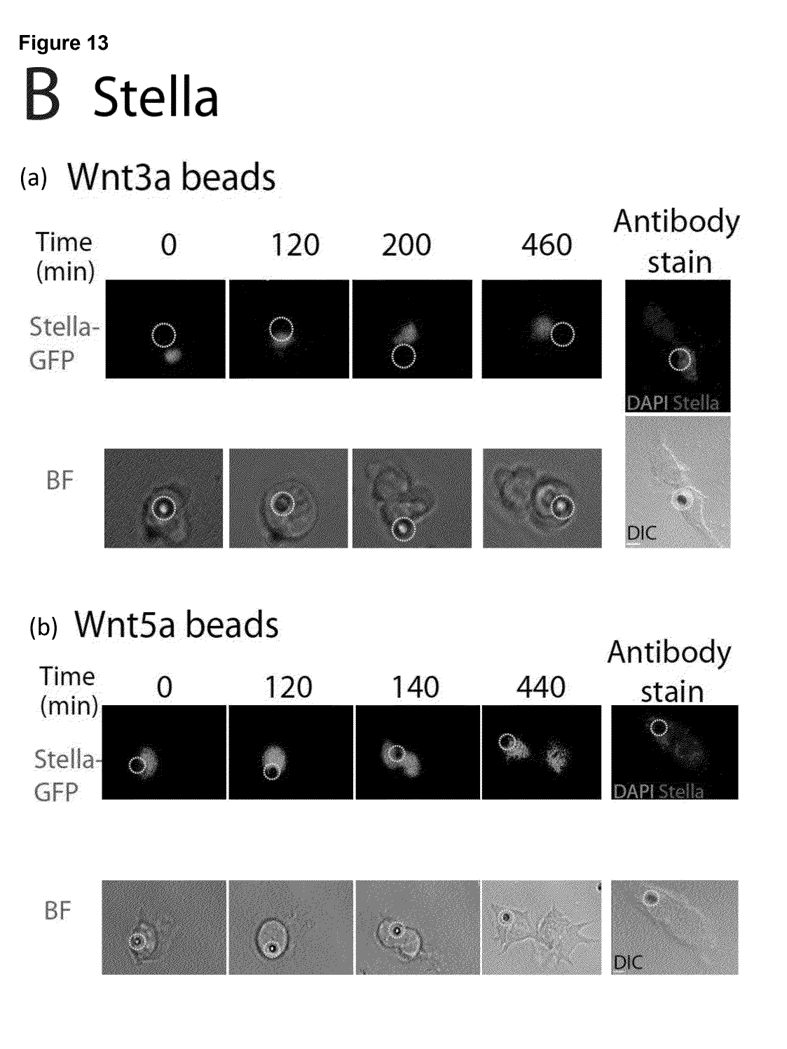

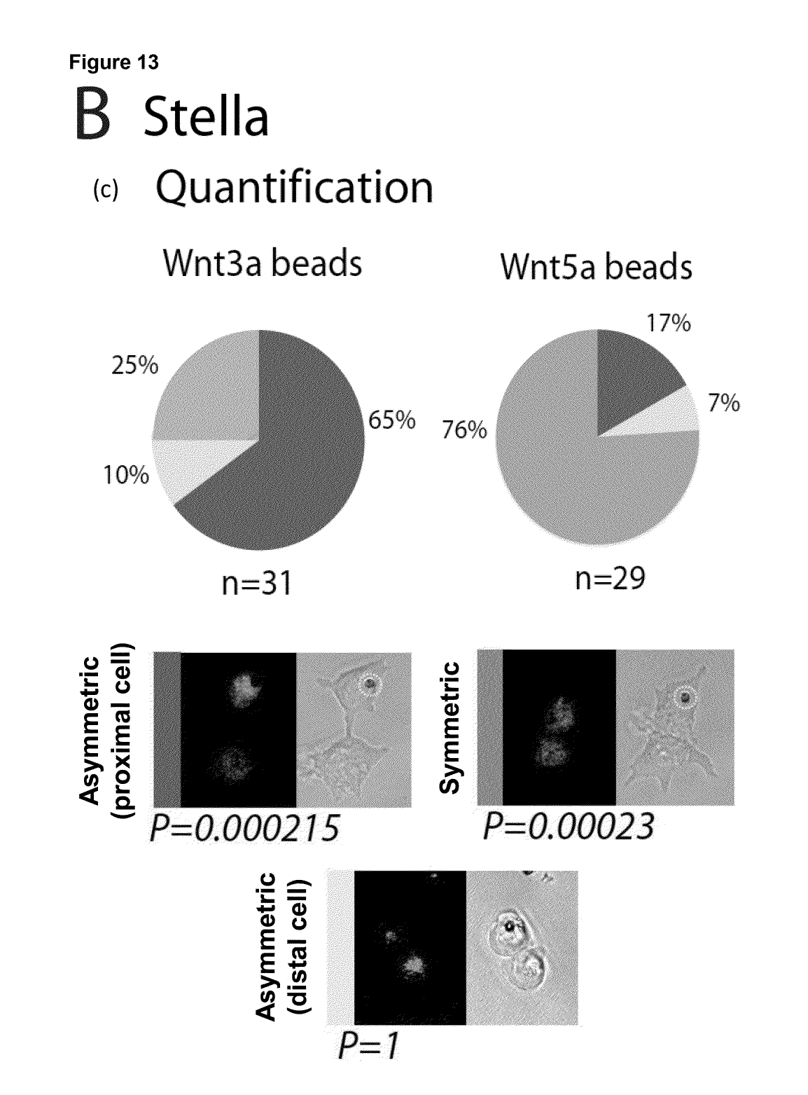

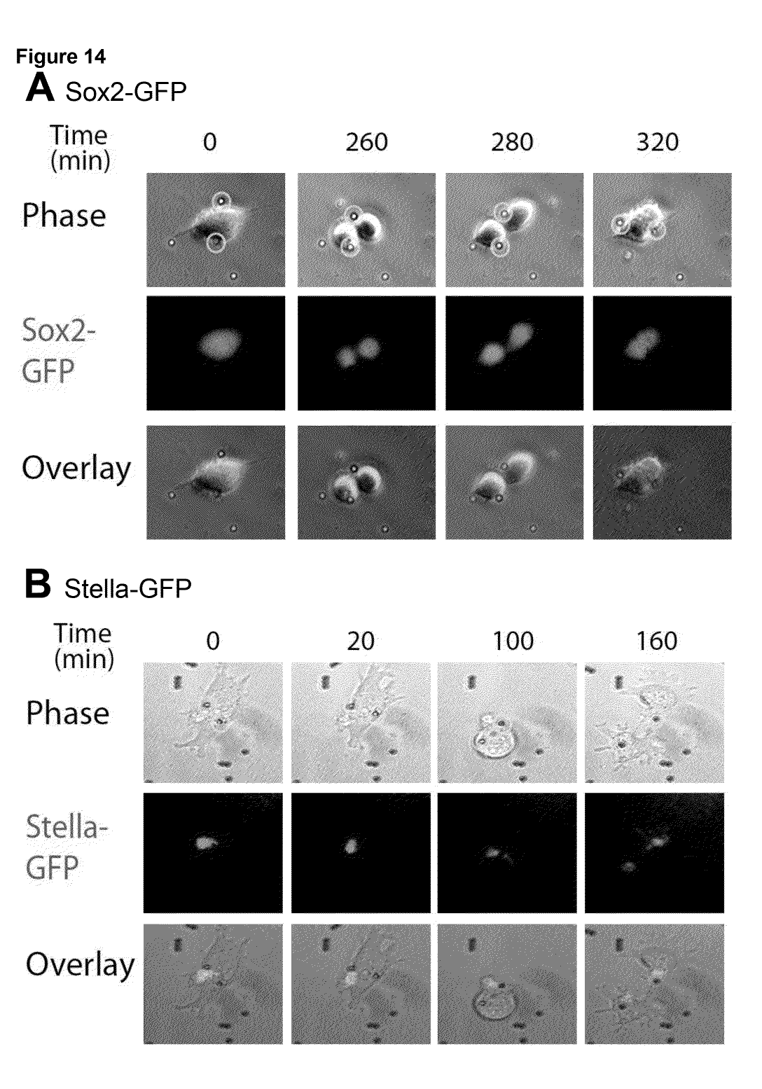

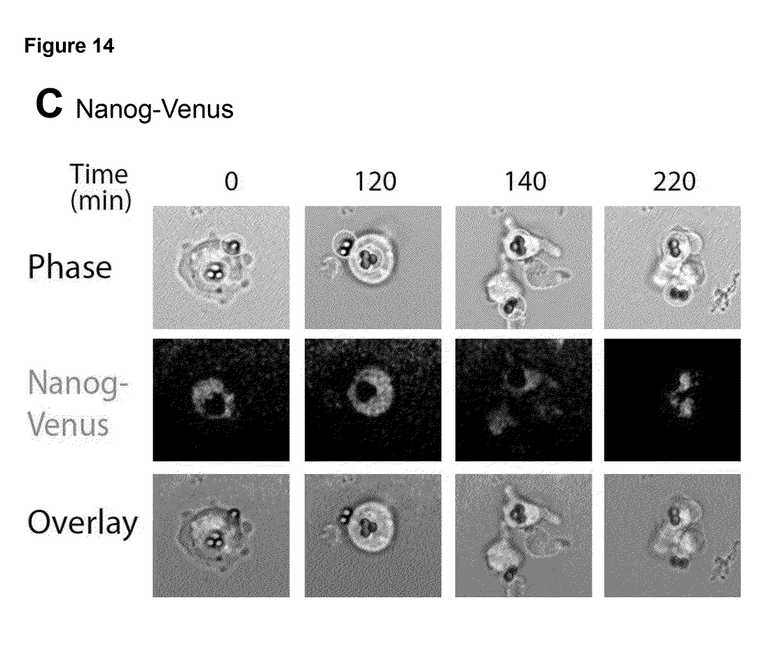

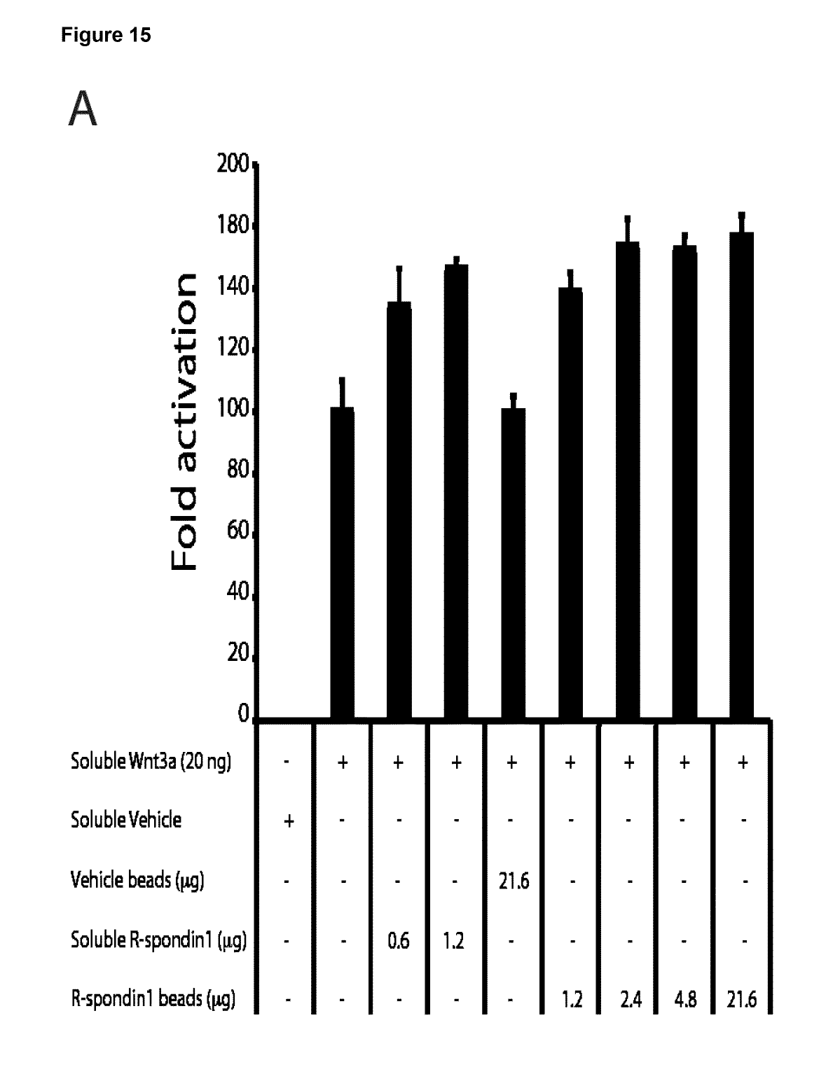

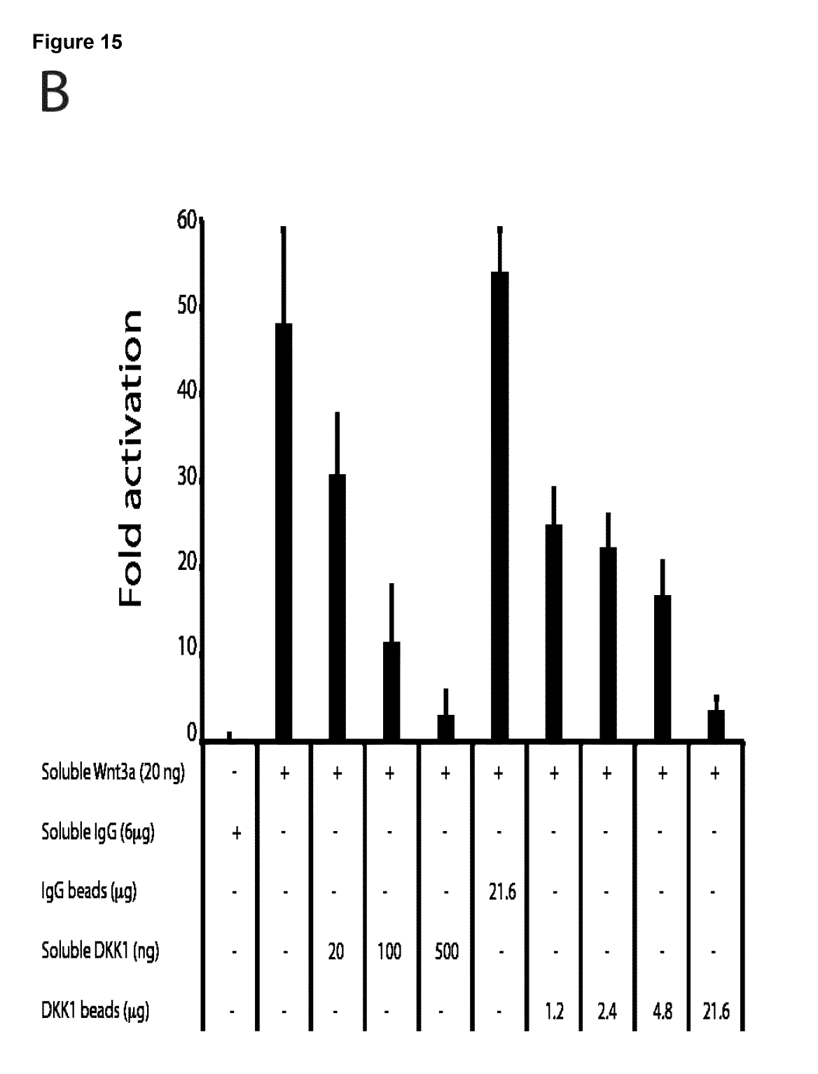

We investigated the effect of localized Wnt signals on pluripotency gene expression by using various ES reporter cells, including cells expressing a Nanog-Venus fusion protein and GFP-based reporters for Rex1, Sox2 and Stella. Pluripotency proteins were also followed by antibody staining. The expression of Nanog, Rex1 and Stella has been shown to decline during ES cell differentiation. In dividing ES cells, we found that the transcriptional activity and the protein level of the pluripotency markers were markedly higher in the Wnt3a proximal daughter cell compared to the distal cell (FIG. 11A and C; FIG. 12A-C; FIG. 3A and E; FIG. 13). Different expression levels were detectable before cytokinesis was complete. In contrast, in the presence of Wnt5a beads, the daughter cells had similar levels of the markers (FIG. 11B and C; FIG. 12D-F; FIG. 3B and E; FIG. 13). As might be expected, cells exposed to two Wnt3a beads at opposing ends divided symmetrically (FIG. 14). As additional controls, we generated attenuated forms of Wnt3a, using Wnt3a beads that were treated after coupling with a range of concentrations of the reducing agent Dithiothreitol (DTT), lowering the signaling activity of the beads in a dose-dependent manner (FIG. 7B and C). The potency of these beads in inducing asymmetric gene expression of the reporter Rex1-GFP was reduced commensurate with the remaining level of Wnt signaling (FIG. 10). We also tested the activity of another signal implicated in the Wnt pathway; an active form of R-Spondin bound to beads (FIG. 15A). There was no significant effect on asymmetric gene expression (FIG. 3D and E), possibly related to the behavior of R-Spondin in vivo where it acts as a systemic rather than a local Wnt activator.

Under “standard” conditions, including feeder cells which can be source of Wnts or the 2i conditions, ES cells divide mainly symmetrically. We asked whether a global Wnt environment would be required for the symmetrical divisions that ES cells undergo. As a test, we perturbed Wnt signaling locally by applying the Wnt inhibitor Dickkopf (DKK) on beads (FIG. 15B). Under these conditions, we found a significant number of divisions giving rise to asymmetric gene expression in the daughter cells, but in a manner opposite to the Wnt beads. With DKK-beads, the distal cell had higher expression of the pluripotency gene Rex1 than the proximal cell (FIG. 3C and E). Importantly, we could rescue the effect of a local Wnt or Wnt inhibition by incubating the cells at the same time under the 2i conditions, indicating that the asymmetry induced by Wnt3a or DKK beads was not a non-specific perturbation of the cells (FIG. 3E). Based on the asymmetric Wnt inhibition experiments, it is shown that uniform Wnt signaling is required for symmetric daughter cell fate.

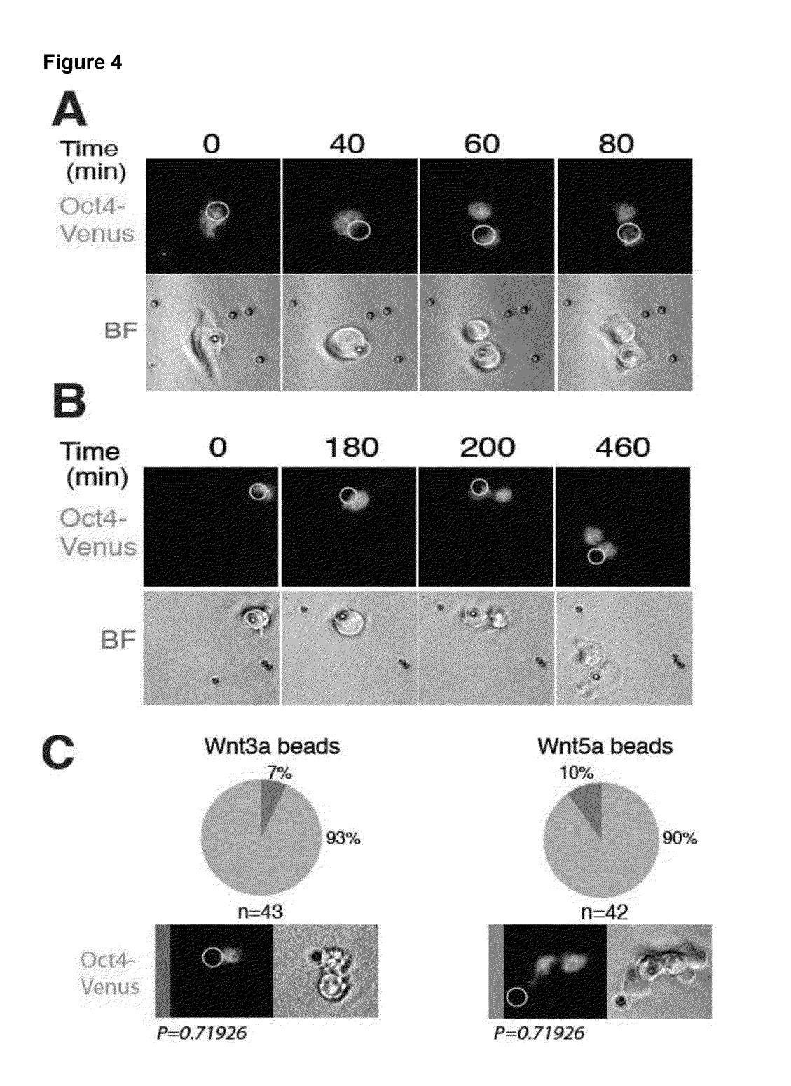

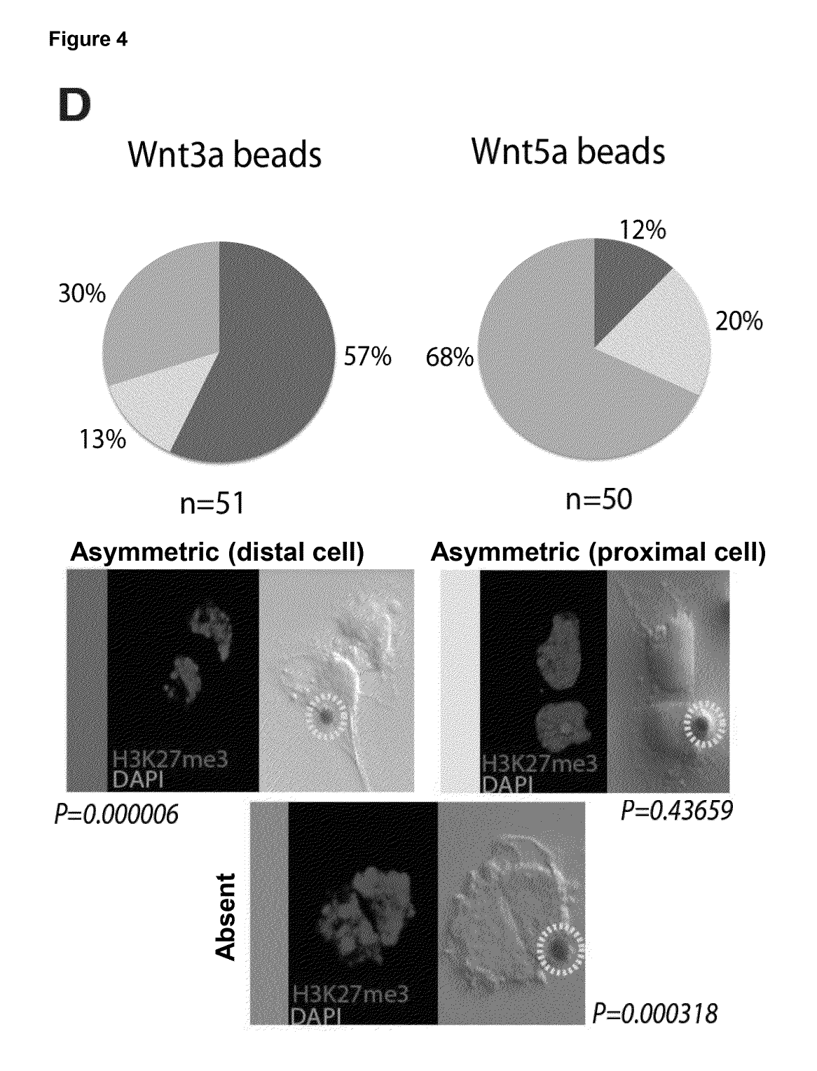

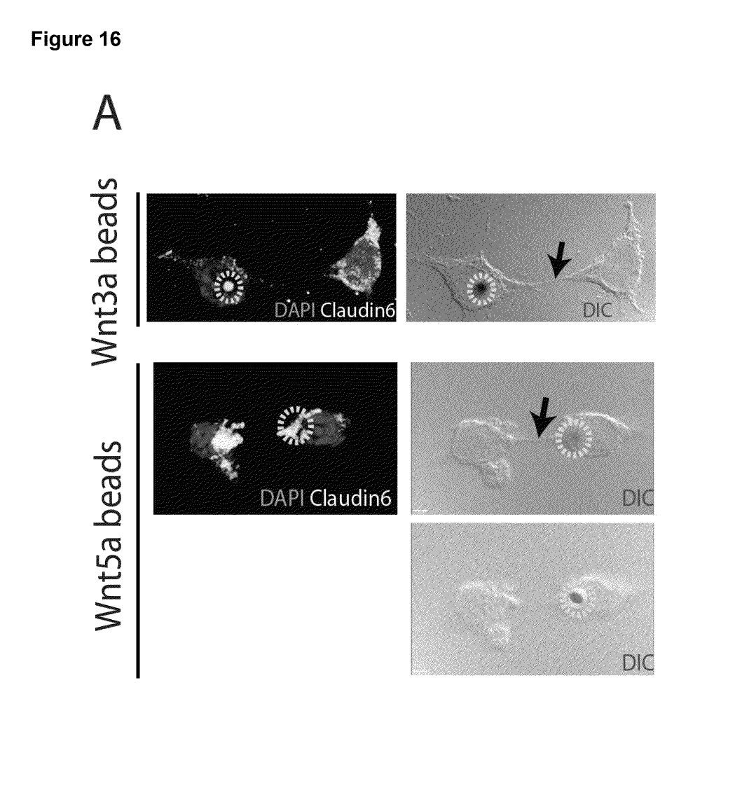

The lower levels of markers of pluripotency in the Wnt3a-distal daughter cell show that distal cells enter a differentiation program with hallmarks of EpiSC. The pluripotency gene Oct4 is expressed at similar levels in ES and EpiSC. We examined Oct4-Venus ES reporter and found symmetrical distribution of Oct4-Venus after cell division, either in the presence of Wnt3a or Wnt5a beads (FIG. 4A-C). We assessed the expression of EpiSC markers, using H3K27me3 focal accumulation as a hallmark of an inactivated X chromosome in female ES cells. H3K27me3 staining was detected in 57% of Wnt3a bead-distal ES cells after division (FIG. 4D). Levels of Claudin6, another EpiSC marker, were higher in 60% of Wnt3a-distal cells but this marker was more rarely (23%) present asymmetrically in Wnt5a-exposed cells (FIG. 16). Thus, localized Wnt3a signal specifies that the Wnt3a-distal cell enters a differentiation program with hallmarks of EpiSC fate.

The findings reported here show a mechanism for external control of asymmetric stem cell division and differentiation. Specifically, a spatially localized Wnt signal orients the mitotic division plane of stem cells. Then, in the dividing cell, the Wnt signal produces an asymmetric distribution of Wnt signaling components, generating a “Wnt-on” proximal cell maintaining ES pluripotency and a “Wnt-off” distal cell differentiating towards an EpiSC cell fate. Therefore, by orienting cell division, the Wnt signal positions the distal daughter cell out of its signaling range, leading to differentiation. By growing single cells exposed to a symmetry-breaking signal we have developed a system that allows for precise real-time examination of processes involved in asymmetric cell divisions.

Neumuller, J. A. Knoblich. Genes Dev 23, 2675 (2009).

Werts, B. Goldstein. Seminars in Cell and Developmental Biology 22, 842 (2011).

Walston et al. Dev Cell 7, 831 (2004).

Goldstein et al. Dev Cell 10, 391 (2006).

Sugioka et al. Cell 146, 942 (2011).

Ten Berge et al. Nat Cell Biol 13, 1070 (2011).

Sato et al. Nat Med 10, 55 (2004).

Ying et al. Nature 453, 519 (2008).

Surani, J. Tischler. Nature 487, 43 (2012).

Mikels, R. Nusse. PLoS Biol 4, e115 (2006).

Bahmanyar et al. Genes Dev 22, 91 (2008).

Yamashita et al. Science 301, 1547 (2003).

Mogensen et al. J Cell Sci 113, 3013 (2000).

Wang et al. Nature 461, 947 (2009).

Bornens. Science 335, 422 (2012).

Yamashita, M. T. Fuller. J Cell Biol 180, 261 (2008).

Planchon et al. Nat Methods 8, 417 (2011).

Singh et al. Stem Cells 25, 2534 (2007).

Chambers et al. Nature 450, 1230 (2007).

Payer et al. Genesis 44, 75 (2006).

Toyooka et al. Development 135, 909 (2008).

Hayashi et al. Cell Stem Cell 3, 391 (2008).

Arnold et al. Cell Stem Cell 9, 317 (2011).

Kim et al. Science 309, 1256 (2005).

Tesar et al. Nature 448, 196 (2007).

Guo et al. Development 136, 1063 (2009).

Plath et al. Science 300, 131 (2003).

Hilliard, C. I. Bargmann. Dev Cell 10, 379 (2006).

Korkut et al. Cell 139, 393 (2009).

### Example 2

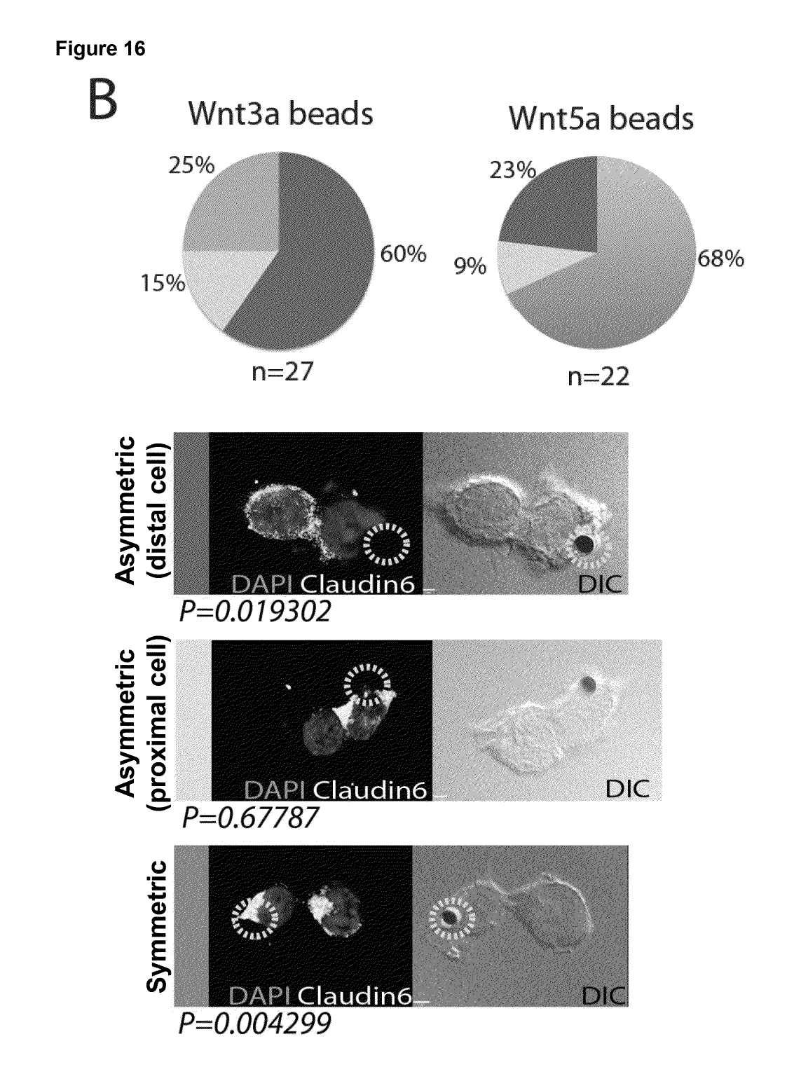

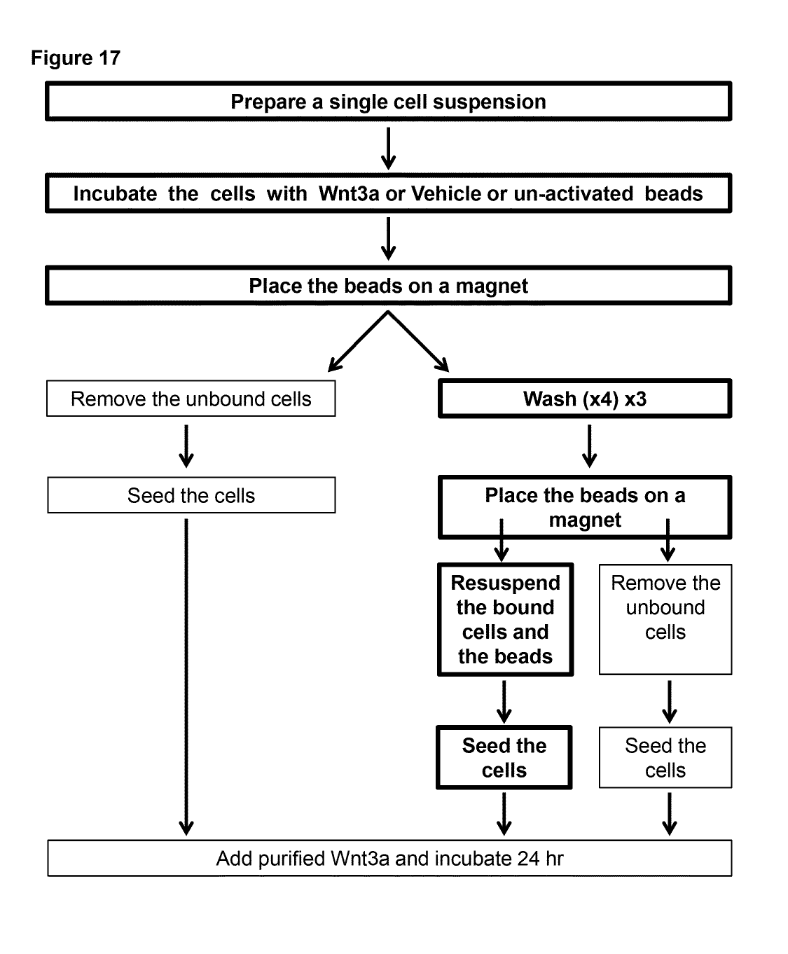

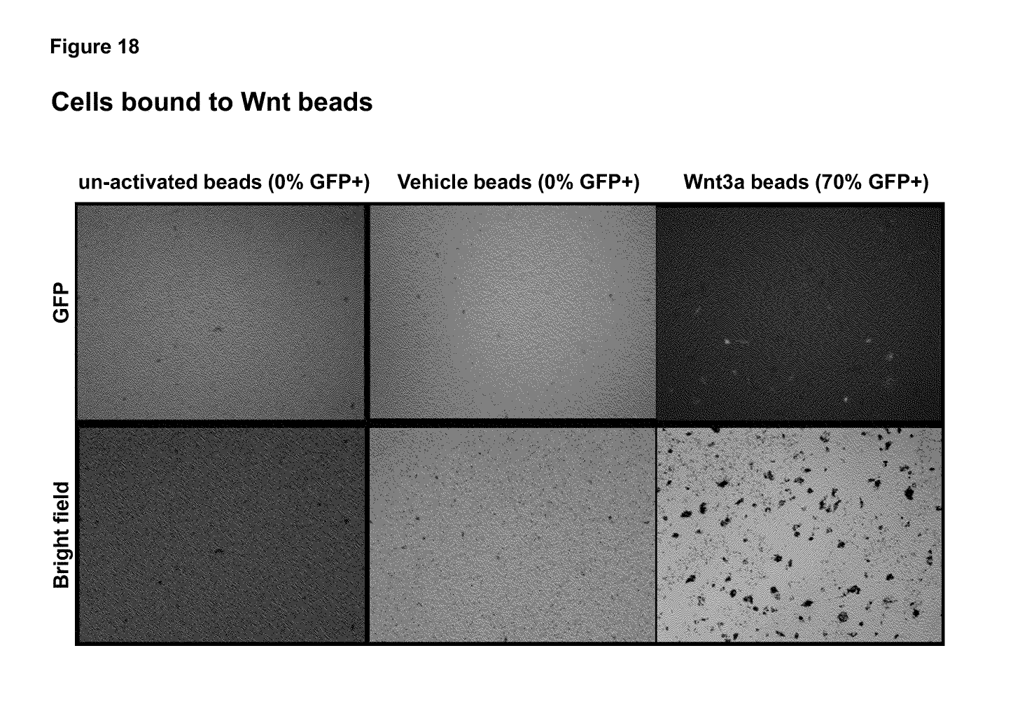

The target cells used in this example were a CommaD beta cell line, a breast progenitor cell line comprised of cells that can give rise to all cell types of the mammary gland. Prior to enrichment, the target cells were transfected with a Wnt reporter (7TCF//eGFP) and 40% were determined to be Wnt responsive via the addition of purified (not bead-coupled) Wnt3a. FIG. 17 depicts the scheme used to enrich the population of target cells for Wnt responsive cells. The percent of isolated cells that were Wnt responsive (GFP+) for each scenario was determined by adding purified Wnt3a (final step of the scheme presented in FIG. 17). As illustrated in FIG. 18, when the target cells were contacted with Vehicle only or with un-activated beads, none of the isolated cells were Wnt responsive (GFP+). When the target cells were contacted with Wnt3a-coupled beads, 70% of the isolated cells were Wnt responsive (GFP+). Thus, Wnt3a-coupled beads were used to enrich a target cell population for Wnt responsive cells (from 40% to 70%). By optimizing the parameters of the scheme presented in FIG. 17, it is predicted that even greater enrichment will be achieved.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of modulating the wnt signaling pathway in a target cell, the method comprising:
contacting the target cell with:
(a) a biologically active wnt stimulator polypeptide coupled to a magnetic bead; or
(b) a biologically active wnt inhibitor polypeptide coupled to a glass or magnetic bead
for a period of time sufficient to stimulate or inhibit wnt pathway signaling activity.

2. The method of claim 1, wherein the wnt stimulator polypeptide is selected from a group consisting of: Wnt, Norrin, R-spondin, and a combination thereof.

3. The method of claim 1, wherein the magnetic bead is positioned within the magnetic field of a magnet such that the location of the magnetic bead is controlled by the location of the magnet.

4. The method of claim 1, wherein the wnt stimulator polypeptide that is coupled to a magnetic bead is 70% or more biologically active when stored in a buffer that is substantially free of detergent.

5. The method of claim 1, wherein the target cell is in vitro.

6. The method of claim 1 wherein the target cell is a pluripotent stem cell (PSC).

7. The method of claim 6, wherein the PSC is selected from the group consisting of:
an embryonic stem cell (ESC), an epiblast stem cell (EpiSC), an induced pluripotent stem cell (iPSC), and an embryonic germ stem cell (EGSC).

8. The method of claim 1, wherein the target cell is in vivo.

9. The method of claim 1, wherein the target cell is an isolated cell that is not in direct contact with a neighboring cell.

10. The method of claim 1, further comprising detecting the presence of wnt pathway signaling activity.

11-12. (canceled)

13. The method of claim 1, further comprising evaluating a cellular effect of the elicited wnt pathway signaling activity.

14-15. (canceled)

16. The method of claim 13, wherein the evaluating comprises determining the absence or presence of a marker of cellular differentiation.

17-18. (canceled)

19. The method of claim 1, wherein the wnt inhibitor polypeptide is selected from a group consisting of: wnt5, wnt11, DKK (Dickkopf), sFRP (Secreted Frizzled Related Protein), WIF (Wnt Inhibitory Factor), and a combination thereof.

20. A method of enriching a target cell population for Wnt responsive cells, the method comprising:
contacting the target cell population with wnt-coupled magnetic beads;
positioning the wnt-coupled magnetic beads within the magnetic field of a magnet to produce an isolated cell population comprising cells that are bound to the wnt-coupled magnetic beads; and
resuspending the cells of the isolated cell population to produce a cell population that is enriched for Wnt responsive cells.

21. The method of claim 20, wherein the wnt-coupled magnetic beads are at least 70% biologically active when stored in a buffer that is substantially free of detergent.

22. A method of producing a biologically active bead-coupled wnt polypeptide, the method comprising:
(a) contacting a bead with exposed carboxylic acid groups with:
(i) carbodiimide;
(ii) NHS (N-hydroxysuccinimide); and
(iii) MES (2-(N-morpholino)ethanesulfonic acid) buffer, to produce an activated bead;
(b) contacting the activated bead with a wnt polypeptide that is at a concentration ranging from 100 ng/μl to 300 ng/μl,
to produce a bead-coupled wnt polypeptide; and
(c) contacting the bead-coupled wnt polypeptide with a buffer that is substantially free of detergent.

23. (canceled)

24. A composition for modulating the wnt signaling pathway, the composition comprising: a biologically active wnt stimulator polypeptide coupled to a magnetic bead, or a biologically active wnt inhibitor polypeptide coupled to a glass or magnetic bead.

25. The composition of claim 24, wherein the wnt stimulator polypeptide is a wnt polypeptide.

26. The composition of claim 25, further comprising a buffer that is substantially free of detergent and the wnt polypeptide is at least 70% biologically active.

27. (canceled)

28. The composition of claim 24 claim 27, wherein the biologically active wnt inhibitor polypeptide is selected from a group consisting of: wnt5, wnt11, DKK (Dickkopf), sFRP (Secreted Frizzled Related Protein), and WIF (Wnt Inhibitory Factor).

29-31. (canceled)
